(19) United States
(12) Patent Application Publication
(10) Pub. No.: US 2021/0369667 A1
(43) Pub. Date: Dec. 2, 2021

(54) COMPOSITIONS AND METHODS FOR THE PREVENTION AND TREATMENT OF RADIATION DERMATITIS, ECZEMA, BURNS, WOUNDS AND CERTAIN CANCERS

(71) Applicant: VIZURI HEALTH SCIENCES CONSUMER HEALTHCARE, INC., Bonita Springs, FL (US)

(72) Inventor: Philip J. BIRBARA, West Hartford, CT (US)

(21) Appl. No.: 17/290,309

(22) PCT Filed: Mar. 27, 2020
(86) PCT No.: PCT/US2020/025270

§ 371 Date: Apr. 30, 2021

Related U.S. Application Data
(60) Provisional application No. 62/826,885, filed on Mar. 29, 2019.

(51) Int. Cl. A61K 31/353; A61K 31/728; A61K 31/7048; A61K 31/12; A61K 31/05; A61K 47/06; A61K 47/46; A61K 31/455; A61K 31/197; A61K 9/00; A61P 17/02

(52) U.S. Cl. CPC A61K 31/353; A61K 31/728; A61K 31/7048; A61K 31/12; A61K 31/05; A61P 17/02; A61K 47/46; A61K 31/455; A61K 31/197; A61K 9/0014; A61K 47/06

(57) ABSTRACT

Novel polyphenol compositions and methods for the treatment and prevention of radiation dermatitis, eczema, thermal burns, wounds, and certain cancers. The disclosure also relates to methods for the preparation of polyphenol compositions including flavonoid compositions.

This application claims priority to U.S. Provisional Application 62/826,885, filed Mar. 29, 2019, the content of which is incorporated herein by reference in its entirety.

The present disclosure relates to polyphenol compositions and methods for the treatment and prevention of radiation dermatitis, eczema, burns, wounds and certain cancers. The disclosure also relates to methods for the preparation of polyphenol compositions including flavonoid compositions.

### BACKGROUND

The goal of radiotherapy is to target radiation exposure to the tumor while limiting exposure of normal tissue. The ionizing effects, reactive oxygen species (ROS), and free radicals produced by radiation can result in damage to cellular DNA, intracellular and cell envelope membrane, and cellular organelles, which can lead to cell death. Although this effect is desirable for eradication of the tumor, which requires that total cell death be achieved, it can be damaging to other tissues, such as skin, which then need to be repaired. Normal tissue has a greater capacity to repair itself than do tumor cells, so it is possible for an effective treatment course to find a way to destroy the tumor while causing only sub-lethal damage to other tissues. Even with the best therapeutic regimen, however, rapidly proliferating normal tissue, such as the basal layer of epidermis, gastrointestinal mucosa, and hematopoietic cells, is at great risk for radiation damage. Since the radiation used in radiotherapy of various cancers typically passes through the skin on its way to the target tumor, skin is often quite susceptible to radiation damage during a treatment regimen of radiotherapy.

In the US, Canada, Europe and Australia at least 50% of patients diagnosed with cancer receive radiation therapy. Approximately 75% of oncology patients receive radiation therapy as a component of curative or palliative care. Up to 95% of patients receiving radiation therapy experience some degree of skin reaction. The skin reactions resulting from irradiation—radiation dermatitis or radiodermatitis—can range from mild to severe and are related to both treatment- and patient-related factors.

### Radiation Dermatitis

In general, a patient's risk for radiation dermatitis is proportional to the degree of skin exposure to radiation. The goal of radiotherapy is to irradiate tumor cells while minimizing damage to normal tissue. However, normal cells in the radiation field can also be damaged by radiation exposure. Typically, normal tissues are capable of self-repair, but repetitive radiation exposure creates an imbalance of tissue damage and repair. In addition to radiation exposure, there are additional patient-related factors include age, nutritional and performance status, weight, radiosensitivity, skin integrity of site, presence of inflammation, lymph drainage and co-morbidities, that play a role in the severity of the radiation dermatitis developed. Each person reacts to treatment in a different way. The likelihood and severity of a skin reaction depends on the area being treated, the type and dose of radiation given, and whether chemotherapy is also given. After 2 or 3 weeks of radiation therapy, skin may become pink or tanned. As treatment continues, skin may become bright red or very dark and may also feel dry and itchy and look flaky. Some people develop a rash or blisters in the treatment area. These blisters may open and peel. Generally, the symptoms and severity of radiation dermatitis are dependent on the accumulated radiation dose (Bray et al., Dermatol Ther (Heidelb) (2016) 6:185-206).

### Per Table 1 of Bray et al.:

TABLE 1
Dose-dependent acute cutaneous findings after local radiation exposure [7]

| Observed acute skin reaction | Radiation dose (Gy) | Onset of findings |
|---|---|---|
| Transient erythema | 2 | Hours |
| Paint erythema and epilation | 6-10 | 7-10 days |
| Defined erythema and | 12-20 | 2-3 weeks |
| hyperpigmentation | | |
| Dry desquamation | 20-25 | 3-4 weeks |
| Moist desquamation | 30-40 | 4 weeks or more |
| Ulceration | >40 | 6 weeks or more |

The grading of radiation dermatitis is summarized in Table 2 from Bray et al. (2016):

TABLE 2
Classification of acute radiation dermatitis
Grade

| 1 | 2 | 3 | 4 |
|---|---|---|---|
| Faint | Moderate to | Moist | Skin necrosis or |
| erythema | brisk erythema | desquamation | ulceration of |
| or dry | or patchy moist | other than skin | full-thickness |
| desquamation | desquamation, | folds; pitting | dermis; may |
| | mostly confined | edema, bleeding | have |
| | to skin folds | from minor trauma | spontaneous |
| | and creases; | or abrasion | bleeding from |
| | moderate | | affected area |
| | erythema | | |

According to National Cancer Institute Common Terminology Criteria or Adverse Events Version 3

Radiation dermatitis also presents in acute and chronic forms. These are summarized in Hegedus et al. International Journal of Dermatology, 56, 909-914 (2017), Table 1:

TABLE 1
Differences between acute and chronic radiation dermatitis

| | Symptoms | Manifestation |
|---|---|---|
| Acute radiation | Prisnary transient erythema | Within 90 d of initiation |
| dermatitis | Generalized erythema | of radiation therapy |
| | Skin dryness | |
| | Hair loss | |
| | Hyperpigmentation | |
| | Dry desquamation | |
| | Skin scaling and flaking | |
| | Moist desquamation | |
| Chronic radiation | Epidermal thinning | More than 90 d after |
| dermatitis | Dermal atrophy | initiation of radiation |
| | Fibrosis | therapy |
| | Edema formation | |
| | Thickening of the dermis | |
| | Dyspigmentation | |
| | Telanglectasia | |
| | Dermal necrosis | |

Up to 95% of subjects undergoing radiotherapy develop varying degrees of dermatitis within 4 to 5 weeks of active treatment. Approximately 87% of oncology patients will experience moderate to severe radiodermatitis during or after their therapy. Radiodermatitis can, as above noted, be acute or chronic, and includes localized erythema and edema, skin shedding (desquamation), hair loss (epilation), fibrosis, and necrosis. Radiodermatitis can be painful and embarrassing and has been associated with decreased quality of life and delays in treatment.

Radiation effects can begin with transient erythema occurring after only 1-3 days of radiation therapy due to capillary congestion and dilation. Erythema typically begins occurring after doses of 20-40 Gray (Gy) (week 2-4) and is associated with histamine release and increased blood volume because of dilated post-capillary venules. Dry desquamation with or without pruritus begins occurring at 45 Gy (week 4-5) and moist desquamation begins occurring at 45-50 Gy (week 4-6). Moist desquamation is characterized by blistering, peeling, weeping and sloughing of the skin.

Acute skin reaction is one of the most common side effects of radiation therapy. The severity of radiation skin reaction is graded on a continuum ranging from erythema and dry desquamation to the more severe moist desquamation and, eventually, ulceration. When dry desquamation occurs, there is no skin breakdown and no potential for infection. When moist desquamation occurs, the skin becomes open and susceptible to infection. There is no standard of care so practices differ between institutions and also between individual practitioners.

About 90% of patients treated with radiation therapy for breast cancer, for example, develop radiation—induced dermatitis, which can cause significant patient discomfort, disruption to daily life, and treatment interruption. (Harper J L, et al., Skin toxicity during breast irradiation: pathophysiology and management. South Med J 2004; 97:989-993. [15558927]). The severity of skin reactions during and following breast irradiation is influenced by both treatment-related factors and patient-related factors. Treatment-related factors include the fraction size (the dose delivered with each treatment), the total dose delivered, the volume of tissue treated, the type of radiation and the addition of chemotherapy.

Although many studies have evaluated the role of topical creams and lotions for the prevention or treatment of radiation dermatitis, there is no accepted therapeutic standard of care. Common recommendations at the onset of therapy include measures to keep skin in the treatment area clean and dry; others may include the use of non-scented soaps to minimize skin irritation. Guidelines for skin care during radiotherapy are often aimed at preventing further exacerbation of radiation dermatitis that has already developed.

There is presently no known evidence-based optimal treatment for radiation dermatitis. Despite the high number of trials in this area, there is limited good, comparative research that provides definitive results suggesting the effectiveness of any single intervention for reducing radiation induced skin reactions. Chan, R. J. et. al., Prevention and treatment of acute radiation-induced skin reactions: a systemic review and meta-analysis of randomized controlled trials, BMC Cancer, 2014, 14:53. Bolderston, N. S., et al., The prevention and management of acute skin reactions related to radiation therapy, Cancer Care Ontario, Feb. 21, 2005.

Numerous “lotions and potions” have been suggested for the prevention and management of radiodermatitis, such as ascorbic acid, vitamin D, aloe vera, chamomile and calendula creams, and almond ointment. In fact, mild soap and water has been found to be more effective in the prevention and treatment of radiodermatitis than topical aloe vera. Richardson, J. et al, Aloe vera for preventing radiation-induced skin reactions: A systematic literature review. Clinical Oncology, 17, 478-84, 2005.

Many of today's practices have evolved historically and often lack supporting empirical evidence. A study by Burch however, that tested a variety of commonly used skin care products and deodorants, concluded that skin reactions should not increase if these agents are applied during treatment and that there is no significant bolus effect with normal use of the products. (Burch et al, Measurement of 6-MV x-ray surface dose when topical agents are applied prior to external beam irradiation. Int J Radiat Oncol Biol Phys. 1997; 38:447-51.) An investigative survey by D′haese et al. (2005) found that there is wide discrepancy between nursing interventions for the prevention and management of radiodermatitis. D′haese et al. interviewed radiation oncology nurses in Belgium and found only a small to moderate level of agreement between nurses regarding the prevention and management of radiodermatitis. Greatest variation was between preventative practices, suggesting confusion among oncology nurses (and likely their patients) in regards to the prevention and management of radiation dermatitis.

In a single-blind trial, 50 women receiving radiation therapy for breast cancer were treated with either chamomile or placebo. Chamomile failed to prove superior to placebo for preventing skin inflammation caused by the radiation therapy. Chamomile contains many flavonoids including apigenin, luteolin and quercetin that contribute to its medicinal properties. Maiche A G, et al., Effect of chamomile cream and almond ointment on acute radiation skin reaction. Acta Oncol. 1991; 30:395-396.

The efficacy of an aloe vera gel, a therapy commonly used to prevent radiation skin toxicity, has been evaluated recently in two randomized trials. Williams et al conducted two trials involving women receiving breast irradiation that compared skin toxicity between those receiving Aloe Vera gel and a control group. The first trial was a double-blinded trail in which 194 women were randomized to receive topical Aloe Vera gel or a placebo. In the second trial, 108 patients were randomized to Aloe Vera or no treatment. The scoring of skin toxicity was similar for both arms of the two trials. This suggests that Aloe Vera has no protective effect for those receiving breast irradiation. (Williams M S et al., Phase III double-blind evaluation of an Aloe Vera gel as a prophylactic agent for radiation-induced skin toxicity. Int J Radiat Oncol Biol Phys 1996; 36:345-349.) Review of 10 trials concluded that topical application of aloe vera did not appear to prevent radiation-induced skin damage (firm conclusions could not be drawn due to methodologic problems). Aqueous cream was shown to be significantly more effective than aloe vera gel in reducing incidence of dry desquamation and moderate to severe pain (Heggie et al 2002).

One intervention that appears to provide a preventative benefit is mometasone furoate (MMF) corticosteroid cream. The efficacy of MMF as a prophylactic and therapeutic intervention was investigated in a randomized trial. Forty-nine patients receiving breast radiotherapy were randomized in a double-blinded placebo controlled trial to receive MMF and an emollient cream or a placebo emollient cream during their radiotherapy and for three weeks following. This trial demonstrated that prophylactic application of MMF combined with an emollient cream significantly decreased acute radiation dermatitis compared with emollient cream alone. (Bostrom A, et al., Potent corticosteroid cream (mometasone furoate) significantly reduces acute radiation dermatitis: results from a double-blind, randomized study. Radiother Oncol 2001; 59:257-265). This outcome, however, must be tempered by the fact that application of emollients at the time of radiation treatment is currently not recommended as emollients have been suggested to increase the severity of radiation dermatitis when used in this way (Bray et al., 2016). Therefore, the emollient control used in this study may have produced a misleading control baseline against which MMF cream was compared.

Other agents that have been used include: calendula, glutathione and anthocyanin, urea lotion (Eucerin), anionic polar phospholipid cream, vitamin C lotion, chamomile cream and almond ointment, and sodium sucrose octasulfate. There are no consistent data suggesting any preventative effect.

Dressing Solutions include hydrocolloid dressings and silver leaf dressings, no sting barrier film (Cavilon), Vaseline impregnated gauze and honey impregnated gauze. These are useful as palliative/symptomatic treatments but do not have demonstrated preventative effects.

Because of the above summarized, general lack of effectiveness of interventions to reduce the incidence of or severity of radiation dermatitis, at the present time commonly used products for management of radiation dermatitis are essentially symptomatic/palliative in function. That is, their mode of action is primarily to protect and facilitate the healing of radiation damaged skin.

The most common products currently used in the treatment of skin exposed to ionizing radiation include: Aquaphor Healing Ointment, Biafine RE (Radiodermatitis Emulsion), Keri Lotion, 100% Aloe Vera Gel, Eucerin and Cetaphil.

Both the Aquaphor and Biafine formulation are recommended by radiation oncologists and dermatologists for the treatment of radiation dermatitis. It is recommended that a small amount of cream be applied to the affected treatment area three times a day, seven days a week. Aquaphor is used as a moisturizer to treat or prevent dry, rough, scaly, itchy skin and minor skin irritations (e.g., diaper rash, skin burns from radiation therapy). Aquaphor Healing Ointment ingredients include Petrolatum, Mineral Oil (Paraffinum Liquidum), Ceresin, Lanolin Alcohol, Panthenol, Glycerin, Bisabolol (L-Alpha).

Biafine is a wound-healing product that has been touted to reduce radiation-related skin toxicity. Biafine RE contains purified water, liquid paraffin, ethylene glycol monostearate, stearic acid, propylene glycol, paraffin wax, squalane, avocado oil, trolamine/sodium alginate, triethanolamine, cetyl palmitate, methylparaben (sodium salt), sorbic acid (potassium salt), propylparaben (sodium salt), and fragrance. The wound-healing properties of Biafine are a result of its capacity to recruit macrophages to epidermal wounds and promote granulation tissue formation. (Coulomb B, et al, Biafine applied on human epidermal wounds is chemotactic for macrophages and increases the IL-1/IL-6 ratio. Skin Pharmacol 1997; 10:281-287). Biafine was compared with best supportive care, which consisted of Aquaphor (Biersdorf, Lindenhurst, N.Y.) and Aloe Vera, in a randomized trial of women receiving breast irradiation. This trial demonstrated no statistical difference in skin toxicity between those receiving Biafine and those treated with best supportive care. (Fisher J, et al. Randomized phase III study comparing best supportive care to Biafine as a prophylactic agent for radiation-induced skin toxicity for women undergoing breast irradiation: Radiation Therapy Oncology Group (RTOG) 97-13. Int J Radiat Oncol Biol Phys 2000; 48: 1307-1310.)

Topical steroids are commonly used to treat radiation-induced skin inflammation. Corticosteroids have been shown to inhibit the up-regulation of the pro-inflammatory cytokine IL-6 in response to ionizing radiation. (Bostrom A, et al., Potent corticosteroid cream (mometasone furoate) significantly reduces acute radiation dermatitis: results from a double-blind, randomized study. Radiother Oncol 2001; 59:257-265).

The general lack of success for interventions intended to be preventative of radiation dermatitis, despite many approaches that have been tried, indicates that this is a complex problem which does not have an obvious solution. The mechanisms whereby radiation passing through the skin causes the skin damage that leads to radiation dermatitis are manifold. They involve oxidative and free radical damage to cellular membranes, DNA, structural proteins, enzymes, and lipids. They also involve ionization of intracellular chemical substances which can also produce damage similar to that listed preceding. The present disclosure addresses these manifold mechanisms in a novel, multipronged approach.

### Eczema

Eczema is a condition where patches of skin become inflamed, itchy, red, cracked, and rough. Blisters may sometimes occur. Different stages and types of eczema affect 31.6 percent of people in the United States. The word “eczema” is also used specifically to talk about atopic dermatitis, the most common type of eczema. “Atopic” refers to a collection of diseases involving the immune system, including atopic dermatitis, asthma, and hay fever. Dermatitis is an inflammation of the skin. Some people outgrow the condition, while others will continue to have it throughout adulthood.
Certain foods can trigger symptoms, such as nuts and dairy.
Symptoms vary according to the age of the person with eczema, but they often include scaly, itchy patches of skin.
Eczema can also be triggered by environmental factors like smoke and pollen. However, eczema is not a curable condition.
Treatment focuses on healing damaged skin and alleviating symptoms. There is not yet a full cure for eczema, but symptoms can be managed.

The symptoms of atopic dermatitis can vary, depending on the age of the person with the condition. Atopic dermatitis commonly occurs in infants, with dry and scaly patches appearing on the skin. These patches are often intensely itchy. Most people develop atopic dermatitis before the age of 5 years. Half of those who develop the condition in childhood continue to have symptoms as an adult. However, these symptoms are often different to those experienced by children. People with the condition will often experience periods of time where their symptoms flare up or worsen, followed by periods of time where their symptoms will improve or clear up.

Symptoms in infants under 2 years old
Rashes commonly appear on the scalp and cheeks.
Rashes usually bubble up before leaking fluid.
Rashes can cause extreme itchiness. This may interfere with sleeping. Continuous rubbing and scratching can lead to skin infections.

Symptoms in children aged 2 years until puberty
Rashes commonly appear behind the creases of elbows or knees.
They are also common on the neck, wrists, ankles, and the crease between buttock and legs.

Symptoms in adults
Rashes commonly appear in creases of the elbows or knees or the nape of the neck.
Rashes cover much of the body.
Rashes can be especially prominent on the neck, face, and around the eyes.
Rashes can cause very dry skin.
Rashes can be permanently itchy.
Rashes in adults can be scalier than those occurring in children.
Rashes can lead to skin infections.

Adults who developed atopic dermatitis as a child but no longer experience the condition may still have dry or easily-irritated skin, hand eczema, and eye problems.

The appearance of skin affected by atopic dermatitis will depend on how much a person scratches and whether the skin is infected. Scratching and rubbing further irritate the skin, increase inflammation, and make itchiness worse.

There is no cure for eczema. Treatment for the condition aims to heal the affected skin and prevent flare-ups of symptoms. Doctors suggest a plan of treatment based on an individual's age, symptoms, and current state of health.

For some people, eczema goes away over time. For others, it remains a lifelong condition.

There are numerous things that people with eczema do to support skin health and alleviate symptoms, such as:
taking lukewarm baths
applying moisturizer within 3 minutes of bathing to “lock in” moisture
moisturizing every day
wearing cotton and soft fabrics, and avoiding rough, scratchy fibers and tight-fitting clothing
using a mild soap or a non-soap cleanser when washing
air drying or gently patting skin dry with a towel, rather than rubbing the skin dry after bathing
where possible, avoiding rapid changes of temperature and activities that make you sweat
learning and avoiding individual eczema triggers
using a humidifier in dry or cold weather
keeping fingernails short to prevent scratching from breaking the skin

There are several medications that doctors often prescribe to treat the symptoms of eczema, including:
Topical corticosteroid creams and ointments: These are a type of anti-inflammatory medication and should relieve the main symptoms of eczema, such as skin inflammation and itchiness. They are applied directly to the skin. If you want to buy topical corticosteroid creams and ointments, then there is an excellent selection online with thousands of customer reviews.
Systemic corticosteroids: If topical treatments are not effective, systemic corticosteroids can be prescribed. These are either injected or taken by mouth, and they are only used for short periods of time.
Antibiotics: These are prescribed if eczema occurs alongside a bacterial skin infection.
Antiviral and antifungal medications: These can treat fungal and viral infections that occur.
Antihistamines: These reduce the risk of nighttime scratching as they can cause drowsiness.
Topical calcineurin inhibitors: This is a type of drug that suppresses the activities of the immune system. It decreases inflammation and helps prevent flare-ups.
Barrier repair moisturizers: These reduce water loss and work to repair the skin.
Phototherapy: This involves exposure to ultraviolet A or B waves, alone or combined. The skin will be monitored carefully. This method is normally used to treat moderate dermatitis.

Even after an area of skin has healed, it is important to keep looking after it, as it may easily become irritated again.

Pollen is one of the many potential triggers of eczema. The specific cause of eczema remains unknown, but it is believed to develop due to a combination of genetic and environmental factors. Children are more likely to develop eczema if a parent has had the condition or another atopic disease. If both parents have an atopic disease, the risk is even greater. Environmental factors are also known to bring out the symptoms of eczema, such as:
Irritants: These include soaps, detergents, shampoos, disinfectants, juices from fresh fruits, meats, or vegetables.
Allergens: Dust mites, pets, pollens, mold, and dandruff can lead to eczema.
Microbes: These include bacteria such as Staphylococcus aureus, viruses, and certain fungi.
Hot and cold temperatures: Very hot or cold weather, high and low humidity, and perspiration from exercise can bring out eczema.
Foods: Dairy products, eggs, nuts and seeds, soy products, and wheat can cause eczema flare-ups.
Stress: This is not a direct cause of eczema but can make symptoms worse.
Hormones: Women can experience increased eczema symptoms at times when their hormone levels are changing, for example during pregnancy and at certain points in the menstrual cycle.

There are many different types of eczema. The above has focused mainly on atopic dermatitis, other types include:
Allergic contact dermatitis: This is a skin reaction following contact with a substance or allergen that the immune system recognizes as foreign.
Dyshidrotic eczema: This is an irritation of the skin on the palms of the hands and the soles of the feet. It is characterized by blisters.
Neurodermatitis: This forms scaly patches of skin on the head, forearms, wrists, and lower legs. It is caused by a localized itch, such as an insect bite.
Nummular eczema: These show as circular patches of irritated skin that can be crusted, scaly, and itchy.
Seborrheic eczema: This forms oily, scaly, yellowish patches of skin, usually on the scalp and face.
Stasis dermatitis: This is a skin irritation of the lower leg usually related to circulatory problems.

### Thermal Burns and Wounds

Two million people in the U.S. each year are treated for thermal burn injuries. One hundred thousand of these patients require hospitalization. Superficial burns heal spontaneously within the first two weeks. Treatment consists of pain relief and topical wound care to relieve pain and prevent infection. Deeper burns will not heal within 2 weeks and may require surgical intervention and should be cared for by a surgical specialist.

The goal of burn care is to prevent infection and obtain a closed injury site. This may be accomplished in minor wounds by applying topical antibacterial creams. A minor wound may also respond well to non-adherent gauze such as Vaseline impregnated gauze.

Principles of wound care include: preventing desiccation (drying) and preventing infection. Large wounds may require attention to nutritional support, pain relief, inhalation injuries and hospitalization.

When a thermal burn occurs, the initial therapy typically includes cooling of the burn injury site. After that, treatment is rendered by the appropriate family practitioner or regional burn center. Common treatment includes fluid administration (urinary output should be 30-50 cc) and a tetanus toxoid booster; 3rd degree burns are treated with topical antibacterial agents and surgical therapy, such as skin grafting, is often contemplated.

Although there are no well-established natural treatments for minor burns, several preliminary studies suggest a few options for reducing pain and speeding healing.

The use of topical agents to alleviate pain and control infection in burn wounds continues to be a subject of widespread interest. Part of this interest has resulted in the development of antibiotic and trace metal creams currently widely in use in burn centers. In addition, a second part of this interest is related to recurrent reports, largely anecdotal, of home treatment stories, when remarkable results were achieved with extracts from aloe vera plants.

Honey is an ancient remedy which has been rediscovered for the treatment of wounds. Many therapeutic properties have been attributed to honey including antibacterial activity and the ability to promote healing. Evidence of antibacterial activity is extensive, with more than 70 microbial species reported to be susceptible.

A series of studies done in India found that a combination of raw honey and gauze was significantly better than conventional types of bandages for superficial burns treated at a hospital. The burns covered with honey healed faster and with less frequent infection than the burns covered with other types of bandages. Other studies of varying quality have also found evidence of benefit.

Infections in burn patients continue to be the primary source of morbidity and mortality. Topical antimicrobial therapy remains the single most important component of wound care in hospitalized burn patients and in burn care facilities. The goal of prophylactic topical antimicrobial therapy is to control microbial colonization and prevent burn wound infection. In selected clinical circumstances topical agents may be used to treat incipient or early burn wound infections. At the present time, silver sulfadiazine is the most frequently used topical prophylactic agent; it is relatively inexpensive, easy to apply, well tolerated by patients, and has good activity against most burn pathogens. In patients with large burns the addition of cerium nitrate to silver sulfadiazine may improve bacterial control. Mafenide acetate has superior eschar-penetrating characteristics, making it the agent of choice for early treatment of burn wound sepsis. However, the duration and area of mafenide application must be limited because of systemic toxicity associated with prolonged or extensive use.

Dryness, flakiness, and pruritus are typical in freshly epithelialized wounds and result from the loss of production of skin oils. Superficial wounds and true partial-thickness wounds will eventually recover the ability to produce skin oils; however, deeper wounds will not. Topical moisturizers, used as needed (often more frequently than twice a day), will relieve most complaints of flakiness and itching. Hypoallergenic moisturizers that are not alcohol based, and contain no perfumes, are preferred.

There are some mild antipruritic creams that can be acquired without prescription, and these creams are applied as infrequently as will allow for control of itching. There are also several choices of topical corticosteroids that are used as anti-inflammatory and antipruritic agents. These corticosteroids, most of which require physician prescription, are applied as prescribed and seldom require prolonged use. Adverse reactions of burning, itching, erythema, and skin and papular rashes have been reported, and topical corticosteroids should be discontinued if any of these symptoms occur. Systemic effects of topical corticosteroids are considered reversible.

Wound healing is a complex physiological process that requires a series of steps, each with several factors up to completion. The sequential phases of healing process are inflammation, proliferation and migration of connective tissue, production of extracellular matrix including collagen synthesis, epithelial cells migration and proliferation leading to neovascularization of wounded tissue. The inflammatory phase includes changes in capillary permeability, transudation and cellular migration leading to second proliferation phase, in which proliferation of fibroblasts, endothelial cells occurs in injured areas. The last is remodeling phase in which cells production is balanced by cell death, collagen production by degradation and absorption and capillary formation by capillary obliteration.

### Non Melanoma Skin Cancers

The drug most often used in topical treatment of actinic keratoses, as well as some basal and squamous cell skin cancers, is 5-fluorouracil, or 5-FU (Efudex®, Carac®, Fluoroplex®, others). It is typically applied to the skin once or twice a day for several weeks.

Chemical therapy with 5-fluorouracil may be useful for actinic keratoses. Giannotti et al suggested in a case report that topical treatment with imiquimod and acitretin is an alternative to surgery. They prescribed imiquimod 5% cream to be applied 3 times weekly in combination with oral acitretin (20 mg/d) for 4-6 weeks. No adverse events were reported during treatment, and the tumors had resolved at the 6-month follow-up visit.

Xeroderma pigmentosum, which is commonly known as XP, is an inherited condition characterized by an extreme sensitivity to ultraviolet (UV) rays from sunlight. This condition mostly affects the eyes and areas of skin exposed to the sun. Some affected individuals also have problems involving the nervous system.

The signs of xeroderma pigmentosum usually appear in infancy or early childhood. Many affected children develop a severe sunburn after spending just a few minutes in the sun. The sunburn causes redness and blistering that can last for weeks. Other affected children do not get sunburned with minimal sun exposure, but instead tan normally. By age 2, almost all children with xeroderma pigmentosum develop freckling of the skin in sun-exposed areas (such as the face, arms, and lips); this type of freckling rarely occurs in young children without the disorder. In affected individuals, exposure to sunlight often causes dry skin (xeroderma) and changes in skin coloring (pigmentation). This combination of features gives the condition its name, xeroderma pigmentosum.

People with xeroderma pigmentosum have a greatly increased risk of developing skin cancer. Without sun protection, about half of children with this condition develop their first skin cancer by age 10. Most people with xeroderma pigmentosum develop multiple skin cancers during their lifetime. These cancers occur most often on the face, lips, and eyelids. Cancer can also develop on the scalp, in the eyes, and on the tip of the tongue. Studies suggest that people with xeroderma pigmentosum may also have an increased risk of other types of cancer, including brain tumors. Additionally, affected individuals who smoke cigarettes have a significantly increased risk of lung cancer.

The eyes of people with xeroderma pigmentosum may be painfully sensitive to UV rays from the sun. If the eyes are not protected from the sun, they may become bloodshot and irritated, and the clear front covering of the eyes (the cornea) may become cloudy. In some people, the eyelashes fall out and the eyelids may be thin and turn abnormally inward or outward. In addition to an increased risk of eye cancer, xeroderma pigmentosum is associated with noncancerous growths on the eye. Many of these eye abnormalities can impair vision.

Researchers have identified at least eight inherited forms of xeroderma pigmentosum: complementation group A (XP-A) through complementation group G (XP-G) plus a variant type (XP-V). The types are distinguished by their genetic cause. All of the types increase skin cancer risk, although some are more likely than others to be associated with neurological abnormalities.

Oral retinoids have been shown to decrease the incidence of skin cancer in patients with xeroderma pigmentosum. This therapy is limited by dose-related irreversible calcification of ligaments and tendons.

### Polyphenols and Flavonoids

Flavonoids have been shown to act as free radical scavengers, anti-oxidants, superoxide anions, and UV absorbers. Flavonoid compounds are also known to be effective in strengthening collagen structures. Further, flavonoids have been shown to exhibit anti-mutagenic, anti-inflammatory, anti-microbial and antiviral effects.

Apigenin, a common dietary flavone present abundantly in common fruits and vegetables, is a non-toxic and non-mutagenic flavone. Apigenin has long been considered to have various biological activities such as antioxidant, anti-inflammatory, anti-angiogenesis and anti-tumorigenic, in various cell types. Polyphenols especially flavones and their derivatives have ketone groups conjugated to aromatic rings which are activated by electron donor substitutes, thus inhibiting energy transfer and suppressing oxidative stress.

Many flavonoids are practically insoluble in water and almost all solvents suitable for pharmaceutical, cosmetic, and food additive formulations, preventing their direct use as components in topical compositions. Thus, there is a need for methods for enhancing the bioavailability of these flavonoids including flavones by utilizing acceptable ingredients for topical and pharmaceutical applications.

Apigenin expresses a broad range of anti-inflammatory activities including suppression of COX-2, PGE2 and NO production and via inhibition of NF kappa B activation (inhibition of TNF alpha- and IL-1beta-mediated inflammatory responses, inhibition of matrix metalloproteinases (collagen-degrading enzymes)). Fortunately, apigenin is a potent anti-inflammatory agent and can minimize the damage caused by unnecessary inflammation. Numerous scientific studies indicate that apigenin is a useful ingredient in formulations addressing sensitive, or chronically irritated skin and neurogenic inflammation. Apigenin has been cited as an agent that prevents infection, reduces inflammation, promotes healing and reduces or prevents scar tissue formation.

Advancements in immunology, including a better understanding of cytokines, inflammation, and the role they play in producing acute and chronic radiation dermatitis, have prompted ongoing investigation of compounds which may prove to be “cytoprotective.” Finally, advances in basic wound care management have been helpful in the treatment of acute and chronic radiation dermatitis.

U.S. Pat. No. 6,753,325 discloses a composition and method for preventing, reducing or treating radiation dermatitis. The composition can include a flavonoid. The composition and method can be employed to prevent, reduce or treat radiation dermatitis caused by a wide variety of types of radiation exposure and is particularly useful for the prevention, reduction or treatment of radiation dermatitis.

U.S. Pat. No. 9,889,098 discloses methods of making and using flavonoid compositions.

U.S. Pat. No. 8,637,569 relates to methods of increasing solubility of poorly soluble compounds.

U.S. Patent Application 2014/0314686 relates to polyphenol compositions and methods of formulating oral hygienic products.

There remains a need in the art for effective compositions and methods to prevent and treat radiation dermatitis.

There remains a need in the art for effective compositions and methods to prevent and treat eczema.

There remains a need in the art for effective compositions and methods to prevent and treat thermal burns and wounds.

It is an objective of certain embodiments to provide topical compositions that, when applied to a skin area, will prevent, reduce or treat dermatitis caused by exposure of that skin area to radiation, which does not cause side effects to a patient treated with the composition(s).

It is a further objective of certain embodiments to provide topical compositions that, when applied to a skin area, will prevent, reduce or treat eczema, thermal burns or wounds which does not cause side effects to a patient treated with the composition(s).

### SUMMARY

A composition for topical administration may comprise:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
   wherein the composition is greater than 70% water, or water and petrolatum, and has a pH of 3-8. The polyphenol is one or more compounds selected from the group consisting of a phenolic acid, a flavonoid, a stibene, a diferuloylmethane, a tannin, and a lignan. Advantageous polyphenols are apigenin, quercetin, luteolin, rutin, naringenin, morin, genistein, kaempferol, curcumin, resveratrol, EGCG and a catechin. The hyaluronic acid is typically a mixture of: 40-90% by wt. of hyaluronic acid having a molecular weight of 8000-15000 D, and 10-60% by wt. of hyaluronic acid having a molecular weight of 800,000-1.2 million D. In advantageous embodiments, the dermatologically acceptable carrier includes petrolatum and or colloidal oatmeal. In other advantageous embodiments, the composition includes vitamins selected from the group consisting of vitamin B3 and vitamin B5, and or a DNA repair agent.

Embodiments also relate to a method of treating radiation dermatitis in a mammal comprising administering topically to the affected area of skin of said mammal a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
   wherein the composition is greater than 70% water, or water and petrolatum, and has a pH of 3-8.

Embodiments further relate to a method of treating eczema in a mammal comprising administering topically to the affected area of skin of said mammal a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
   wherein the composition is greater than 70% water, or water and petrolatum, and has a pH of 3-8.

Embodiments also relate to a method of treating a tumor in a mammal comprising:
a) administering a therapeutically effective dose of ionizing radiation to a tumor in said mammal, and
b) applying to the affected area of the skin a therapeutically effective amount of a composition comprising:
   i) 0.01-15% by wt. polyphenol in a form selected from the group consisting of a concentrate, a salt, and a nanoparticle,
   ii) 0.01-5% by wt. hyaluronic acid, wherein the hyaluronic acid includes a high molecular weight hyaluronic acid and a low molecular weight hyaluronic acid, and
   iii) greater than 70% by wt. of a dermatologically acceptable carrier,
      wherein the composition is greater than 70% water, or water and petrolatum, and has a pH of 3-8. The ionizing radiation is selected from the group consisting of alpha radiation, beta radiation, X-rays, gamma radiation, and flouroscopic radiation, and the radiation is administered 1-7 times per week.

In a still further embodiment, the disclosure also relates to a method of treating a thermal burn in a mammal comprising administering topically to the affected area of skin of said mammal a therapeutically effective amount of a composition comprising
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
   wherein the composition is greater than 70% water, or water and petrolatum, and has a pH of 3-8.

Embodiments also relate to a method of treating a wound in a mammal comprising: administering topically to the wound of said mammal a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
   wherein the composition is greater than 70% water, or water and petrolatum, and has a pH of 3-8.

Lastly, the disclosure also relates to a method of treating a nonmelanoma skin cancer or xeroderma pigmentosum in a mammal comprising administering topically to the affected area of skin of said mammal a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid as well as a low molecular weight hyaluronic acid,
iii) 0.5-5% by wt. 5-fluorouracil, and
iv) greater than 70% by wt. of a dermatologically acceptable carrier,
   wherein the composition is greater than 70% water, or water and petrolatum, and has a pH of 3-8.

### BRIEF DESCRIPTION OF THE DRAWINGS/FIGURES

FIG. 1 shows a graph of the experimental data comparing the 3 treatments for the management of radiation dermatitis.

FIG. 2 shows a graph of dermatitis score data over the scoring period for each test article, POS control and VHS-1 to VHS-20.

FIG. 3 shows a graph of dermatitis score data over the scoring period comparing VHS-4 to competitor products and POS control.

FIG. 4 shows a graph of dermatitis score data over the scoring period for each test article, POS controls (current and prior studies) and VHS-21 to VHS-37.

FIG. 5 shows a graph of dermatitis score data over the scoring period comparing VHS-21, 23, 32, 34, 36, and 37 to POS controls (current and prior studies).

FIG. 6A shows a procedure for conduct of the study in Example 12. FIG. 6B shows the application procedures during the induction of dermatitis phase of the study in Example 12. FIG. 6C shows the procedures during the treatment and scoring phase of the study in Example 12. FIG. 6D shows the application procedures for Days 11 through 17 of the study in Example 12. FIG. 6E shows the Dermatitis and Skin Conditions Grading Systems.

FIG. 6F shows the scoring parameters for Dermatitis and Skin Conditions Grading Systems.

### DETAILED DESCRIPTION

The present disclosure relates to compositions and methods for the treatment and prevention of radiation dermatitis, eczema, thermal burns and wounds, and certain skin cancers. The disclosure includes polyphenol and flavonoid compositions and methods of their preparation and use.

### Compositions

The disclosure is directed to formulations (e.g. a cream or a lotion) that support a patient's skin care needs leading up to, during and post radiation treatment. The disclosure also includes formulations for treatment of eczema, thermal burns and wounds. The formulations moisturize, penetrate; repair/rebuild; have anti-inflammatory, anti-oxidant, anti-microbial and/or anti-viral properties.

The formulations may include:
i) 0.01-20% by wt. polyphenol (e.g. flavonoid) in a form selected from the group consisting of a concentrate, a salt, a chelate, and/or a nanoparticle,
ii) 0.01-15% by wt. hyaluronic acid, wherein the hyaluronic acid includes a high molecular weight hyaluronic acid as well as a low molecular weight hyaluronic acid, and
iii) greater than 70% by wt. dermatologically acceptable carrier,
   wherein the formulation is greater than 70% water, or water and petrolatum, and has a pH of 3-8.

Advantageously, the formulations are greater than 75% by wt. water, or water and petrolatum, or more advantageously, greater than 80% water, or water and petrolatum. The water, or water and petrolatum levels for the formulation are typically 78-85% by wt., and for some embodiments, 81-88% by wt.

In another advantageous embodiment, the pH of the formulation is between 3 and 8, or 4 and 7. In certain embodiments, the pH is 4.5-5.5.

Inflammation is the body's natural reaction to burns, wounds, infection, injury, irritation, etc. Inflammation normally leads to heat, pain, redness or swelling. Inflammation is generally positive as it shows the body has responded to infection, injury, irritation, etc. and is starting to repair any damage that was caused. However, some inflammation is unnecessary and actually damages the body. The inflammatory response must be actively terminated when no longer needed to prevent unnecessary damage to tissues. Failure to do so can result in chronic inflammation, and cellular destruction. The subject formulations are useful in treating inflammation, and the addition of bactericidal and/or bacteriostatic agents (and optionally anti-viral agents) to a topical formulation contribute to maintaining an environment that is conducive to hastening the healing processes.

Anti-oxidants are useful in skincare products. Since oxidation is one of the processes by which materials degrade, anti-oxidants were first widely used as preservatives. Anti-oxidants have beneficial attributes in a wide spectrum. For example, Vitamins C and E which are used in many skin-care products are examples of widely used anti-oxidants which are beneficial as skin healing additives in the formulations.

### Polyphenols and Flavonoids

Polyphenols, including glycone or aglycone form, are useful in the formulations. Included are phenolic acids, flavonoids, stibenes (e.g. resveratrol), diferuloylmethanes (curcumins), tannins, and lignans.

Important flavonoids include flavonols, flavanones, flavanols (e.g. catechins), flavones, anthocyanins, and isoflavones (genistein).

The disclosure relates to compositions such as alkaline, neutral and acidic aqueous solutions containing one or more polyphenols.

Certain embodiments relate to solid compositions or solutions containing one or more plant extracted or synthetic polyphenols such as curcumin, resveratrol, or flavonoids e.g., flavones, flavonols, flavanols, proanthocyanidins, dihydroflavonols, aglycone flavonoids, apigenin, luteolin, terpenes, chrysin, quercetin, hesperitin, naringin, genistein, daidzein, epigallocatechin gallate, catechin and derivatives of and/or mixtures of one or more flavonoids. Apigenin, quercetin, luteolin, rutin, naringenin, morin, genistein, kaempferol, curcumin, resveratrol, EGCG or other polyphenols can be used in the formulations.

### Apigenin

Recent research indicates that apigenin is a unique flavonoid with a broad range of useful activity. Apigenin is a potent anti-oxidant, anti-irritant and anti-allergen. It also protects the skin from the damaging effects of sun exposure by a unique mechanism. Apigenin has the following structure:

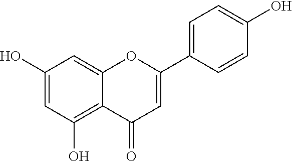 [SMILES: O=c1cc(-c2ccc(O)cc2)oc2cc(O)cc(O)c12]

Apigenin expresses a broad range of anti-inflammatory activities (substances which prevent unnecessary inflammation in the body) including suppression of COX-2, PGE2 and NO production and via inhibition of NF kappa B activation (inhibition of TNF alpha- and IL-1beta-mediated inflammatory responses, inhibition of matrix metalloproteinases (elastin and collagen-degrading enzymes). Fortunately, apigenin is a potent anti-inflammatory agent and can minimize the damage caused by unnecessary inflammation. Apigenin is a useful ingredient in formulations addressing sensitive, or chronically irritated skin and neurogenic inflammation. It prevents infection, reduces inflammation, promotes healing and reduces or prevents scar tissue formation.

Several studies have indicated that apigenin prevents the breakdown of hyaluronic acid by inhibiting the enzyme hyaluronidase; in fact, apigenin is often used as a positive control in hyaluronidase assays. The importance of hyaluronic acid for the cosmetics industry is widely accepted. Hyaluronic acid is also involved in cell-cell and cell-matrix interactions.

Fotsis et al recently showed that apigenin was a potent inhibitor of in vitro angiogenesis. Since angiogenesis is induced by a variety causes including chronic inflammation and because angiogenesis proceeds, in part, through the breakdown of the extracellular matrix and migration of endothelial cells, it is clear that apigenin inhibits angiogenesis at several stages. Apigenin should be considered for formulations addressing spider veins and telangiectasia. Fotsis T, et al., Flavonoids, dietary-derived inhibitors of cell proliferation and in vitro angiogenesis, Cancer Res, 57, 2916-21 (1997).

The compositions include a polyphenol (e.g. flavonoid and/or flavonoid derivative) that have radioprotective effects. In addition, the polyphenols such as the apigenin, cucumin, luteolin, quercetin, catechins etc. have other beneficial effects such as anti-inflammatory, anti-oxidant, anti-bacterial and other desirable properties to assist in the prevention, minimizing and/or preventing the detrimental effects of radiation dermatitis.

The compositions comprise a polyphenol (e.g. flavonoid) and a carrier. Typically, the solubility in water of the flavonoid is less than 1 mg/ml, or less than 0.1 mg/ml. The microparticulate flavonoid has an average size of 200-500 nanometers, or advantageously has an average size of 250 nanometers. In one embodiment, the composition is a pharmaceutical composition and said carrier is a pharmaceutically acceptable carrier. The composition can include hyaluronic acid (sodium hyaluronate), and the carrier typically includes a compound that prevents or reduces agglomeration of the microparticles, a dispersant and/or a penetration enhancer. In one embodiment, the composition is in the form of a colloid, nanosupension or emulsion.

The compositions include one or more polyphenols, such as a flavonoid and/or flavonoid derivative that have radioprotective effects. In addition, flavonoids and/or flavonoid derivatives such as flavones have other beneficial effects such as anti-inflammatory activity and maintaining structural integrity of ischemic or hypoxic tissue, which occur after radiation exposure.

Advantageous combinations of polyphenols include:
i) apigenin and curcumin,
ii) apigenin and narringenin or luteolin,
iii) apigenin and resveratrol

In an advantageous embodiment, the teachings are applicable to poorly soluble polyphenols/flavonoids having a solubility in water less than 1 mg/ml, and particularly less than 0.1 mg/ml.

Advantageous forms of the polyphenols of the compositions are:
i) salts (prepared using the salt technology),
ii) ii) concentrates (prepared using the thermal technology) and,
iii) iii) nanoparticles (prepared using the nanotechnology).

### Salt Technology

The salt technology utilized in certain embodiments is described in U.S. Patent App. No. 62/050,650, the entire contents of which is incorporated by reference herein. The salt technology relates to the formulation of an aqueous soluble alkali metal polyphenol, e.g. flavone salts. The soluble alkali flavone ingredients (including but not limited to the sodium salt of apigenin) comprise 0.01 wt/wt to 5 wt/wt. %, 0.1 wt/wt. % to 5 wt/wt. %, or from 0.3 wt/wt. % to 3 wt/wt. % of the composition. Flavone salt formulations can be formed by adding a flavone to a composition having a preexisting high pH, or alternatively mixing a flavone into a composition having a lower pH and then increasing the pH of the composition by adding an alkali metal hydroxide such as sodium hydroxide to the composition to increase the pH to 7.5 to 11 and thereby convert the flavones to the more aqueous soluble flavones salt form. Included are the sodium, aluminum and potassium salts of the polyphenols.

### Thermal Technology

The thermal technology utilized in other embodiments is described in commonly owned U.S. Pat. No. 8,637,569, the entire contents of which is incorporated by reference herein. For instance, a suitable method for substantially increasing the solubility concentrations of poorly soluble ring structured organic compounds, such as a polyphenol or flavonoid, with heat stable non-toxic solubilizing compounds, such as nonionic surfactant compounds, including polysorbates, comprises the steps of: a) mixing a ring structured organic compound, such as a flavonoid, in a heat stable solubilizing compound to form a mixture; b) heating the mixture while stirring to a temperature where the ring structured organic compound particulates dissolve and the resulting mixture (the “concentrate”) forms a clear solution; and c) cooling the concentrate; and optionally adding a carrier. The mixture is heated to an elevated temperature of greater than for example 100° C., 120° C., 150° C., or 170° C. The temperature selected is that which allows the organic molecules to dissolve to form a solution. The mixture can be heated to a temperature not exceeding the boiling point or decomposition point of either the organic compound or the solubilizing compound. The heating step is advantageously done with only the poorly soluble compound and the solubilizing compound present. The carrier (formulation vehicle components) is advantageously not present during initial mixing or heating. In many embodiments, the ratio of poorly soluble molecule to solubilizing compound approaches 1 mole of planar compound to 2 moles of solubilizing compound, e.g. surfactant for certain combinations. Significantly more surfactant than 1 mole active agent to 2 moles of surfactant is required for some active agent/surfactant combinations, e.g. 1 mole active agent to 20 moles of surfactant. Another embodiment is the step of adding after step b) or c) an alcohol such as ethyl alcohol to the concentrate to form a soluble compound solution with a reduced viscosity. Other advantageous materials to reduce the viscosity level of the solubilized compound mixture include: small-chain alcohols (such as isopropyl and benzyl alcohol), ethoxydiglycol (diethylene glycol monoethyl ether or Transcutol), propylene glycol, hexylene glycol, butylene glycol, dipropylene glycol, glycerin, water, saline, DMSO, isopropyl myristate, mineral oil, low viscosity surfactants, and dimethyl isosorbide.

Apigenin/Polysorbate 80 formulations can be made as follows: Apigenin powder and polysorbate 80 are mixed in the ratio from approximately 5 to 10 wt % of apigenin to 95 to 90 wt % polysorbate 80. A small quantity (5-10 wt %) of DI, water and optionally acetone and/or ethyl alcohol is optionally added to facilitate the blending of the mixture. This mixture is thoroughly stirred to form a thick paste-like blend. The mixture is then slowly heated to relatively high temperatures (about 100 to 150° C.) while stirring. The heating is accompanied by the boiling off of the water and also volatile constituents present in the Polysorbate 80. Upon the removal of the volatiles and heating to temperatures in excess of about 200 to 300 C, a dark brown transparent liquid results such that all the solid apigenin is dissolved in the resulting Polysorbate 80 mixture or “concentrate.” Upon cooling to ambient temperatures, a thick viscous brown liquid results. It is believed that the higher the apigenin content—the darker the resulting color. Based on a 4.05% concentration of apigenin in the viscous apigenin polysorbate 80 concentrate, the content of apigenin is 40.5 mg/ml or 40, 500 parts per million. The use of the apigenin/polysorbate 80 concentrate in an alcohol or aqueous solution can increase bioavailablity—deliver apigenin and other relatively insoluble flavonoids or polyphenols to the desired target location, following administration.

### Nanotechnology

The nanotechnology utilized is described in U.S. Pat. No. 9,899,098, the entire contents of which is incorporated by reference herein. This technology includes “hydrated” microparticulate/nanofiber flavonoid and a carrier. The microparticulate flavonoid can have an average size of 200-500 nanometers, or advantageously can have an average size of 250 nanometers. Most advantageously, the size is 45-90 nm. The compositions can include hyaluronic acid, and the carrier can include a compound that prevents or reduces agglomeration of the microparticles, a dispersant or penetration enhancer.

A hydrated flavonoid or flavone can be formed by: mixing a flavonoid with an alkali metal component (e.g., alkali metal hydroxide(s) and/or alkaline metal salt(s)) to form an alkali metal flavonoid salt; adjusting (e.g., acidifying) the alkali metal flavonoid salt with an agent (e.g., an acidic agent) to a pH level of less than or equal to 7.5 resulting in a gel like precipitate of the flavonoid; filtering out the hydrated flavonoid; and washing of the hydrated flavonoid (e.g., with water such as distilled water) to remove alkaline salts and excess acidifying agent; and, optionally, drying of the hydrated flavonoid. See Examples below.

Fine submicron particles can be formed by control of the acidification process. This includes the rapid addition and mixing of the acidifying agents with the alkaline flavonoid salt solutions until the microparticulates are uniformly distributed. The mixing of the acidifying agent, at temperatures advantageously from 1 to 10° C., with the alkaline salt solution is done such that the ratio of mixing time to precipitation time is minimized (advantageously, a ratio of 1:15, and most advantageously, a ratio of 1:1-2). These ratios contribute to increasing the rate of nuclei formation and limits the rate of crystal growth. The microparticulate hydrated flavonoid can have an average size of 50-1000, advantageously 200-500 nanometers, e.g. averaging 250 nm. Exemplary flavones include, for example, apigenin, luteolin, or a combination thereof. Thus, the method can prepare hydrated flavones including hydrated apigenin, hydrated luteolin, or a combination thereof. Exemplary alkaline metal hydroxides include, for example, sodium hydroxide (NaOH), potassium hydroxide (KOH), lithium hydroxide (LiOH) and calcium hydroxide (Ca(OH)₂) as well as combinations comprising at least one of the foregoing hydroxides. Exemplary alkali metal salts include, for example, citrates (e.g., sodium citrate, potassium citrate, lithium citrate), and carbonates (e.g., sodium carbonate, potassium carbonate, lithium carbonate), as well as combinations comprising at least one of the foregoing salts. Exemplary acids include citric acid and HCl.

Generally, the polyphenol (e.g. flavonoid) compositions can comprise greater than or equal to 0.01 weight percent (wt %) polyphenol, specifically, greater than or equal to 1 wt % polyphenol, for example, 0.1 wt % to 10 wt %, specifically, 0.5 wt % to 8 wt %, more specifically, 2 wt % to 5 wt %, based upon a total weight of the composition. The formulations can comprise greater than or equal to 0.01 wt % polyphenol (flavonoid) (e.g., 0.01 wt % to 20 wt % flavonoid, specifically, 0.05 wt % to 15 wt % flavonoid, more specifically, 0.1 wt % to 10 wt % flavonoid, yet more specifically 0.5 wt % to 4 wt % flavonoid, and even more specifically, 1 wt % to 2 wt % flavonoid), based upon a total weight of the formulation.

### Hyaluronic Acid (HA)

Hyaluronic acid plays an important role in tissue dehydration, lubrication and cellular function. It is produced in the body naturally, however, over time, as with all vitamins and nutrients in the body, the synthesis of hyaluronic acid diminishes. In fact, the half-life of hyaluronic acid in the cartilage is 2-3 weeks, and only 1 day in the skin. Ironically, the molecule itself has a short lifespan and must be frequently produced by cells to replace lost hyaluronic acid. With age, the production of HA declines, which may contribute to many of the diseases that are commonly associated with aging. In fact, hyaluronic acid is found throughout the body, as it forms part of the structural connective tissue that holds together tissues. HA is a fundamental component of the extracellular matrix, which occupies the area between cells. Almost half of the body's HA is located in the collagen of the skin, acting as a moisture-retaining gel.

Within the dermal structure, HA functions as a space filling, structure stabilizing, and cell protective molecule with remarkable malleable physical and superb biocompatibility properties. Additionally, HA structures, which have a high level of visoelasticity, serve to preserve a high level of hydration with this skin. A strong correlation exists between the water content in the skin and levels of HA within the dermal tissue. It is well documented that there are significant alterations in HA physical and biological properties as skin ages—particularly in metabolism, content and deterioration in the mechanical properties of the skin. It is believed that the maintaining of a viable HA presence within the skin's intercellular structure contributes to the viability of a healthy skin physical appearance.

In another aspect, it has been well documented that polysaccharide molecules such as HA do degrade as a consequence of enzymatic and oxidative (free radical) mechanisms. Consequently, it is desirable to develop topical formulations that serve to prevent the decomposition of polysaccharides such as HA. To this end, flavonoids such as flavones serve to meet this need via their well-documented anti-hyaludonidase and anti-oxidant properties—thereby serving to maintain the viability of HA desirable functions protecting against the mechanisms which contribute to its breakdown.

Further, the addition of HA to flavonoid particulate formulations serves to inhibit particulate agglomeration by enhancing the zeta potential of the nanoparticles. Additionally, HA enhances the viscosity of topical formulations thereby serving to prevent nano-particulate stratification and agglomeration.

Topically, HA has water storing properties, making it beneficial as a swelling agent and lubricant, enabling its incorporation into cosmetics leading to a perceptible and visible improvement of skin condition. In use, it forms a thin transparent visco elastic surface film that helps to preserve the characteristics of youthful and healthy skin: suppleness, elasticity and tone. Increased skin hydration may swell and open up the compact structure of the stratum corneum, leading to an increase in penetration of the active flavonoids ingredients of the topical formulations described herein

Scientific studies have shown that hyaluronic acid improves skin hydration, works as an antioxidant and free-radical scavenger, and stimulates the production of collagen in skin. HA also keeps tissues elastic, protecting joints from repeated stresses. Other studies have shown that hyaluronic acid has anti-bacterial and anti-inflammation properties.

Typically, the HA content of the formulation is 0.01-15% by wt., advantageously 0.5-3% by wt.

In an advantageous embodiment, the formulations include more than one molecular weight hyaluronic acid ingredient, for example a low and high MW HA, or three, or even more MW HA ingredients.

Smaller “low” molecular weight hyaluronic acid molecules (MW less than 800 kD including 100-300 kD, 20-50 kD, 8-15 kD and 5-20 kD) have more efficient skin penetration into the dermis and epidermis.

Larger “high” molecular weight hyaluronic molecules (MW greater than 800 kD, typically 800-1,200 kD) provide an occlusive film on the skin's outer surface which helps to retain water in the extracellular matrix of the skin.

Advantageous combinations of low and high MW include HA include: ⅔ by wt, 8-20 KD MW NaHA, and ⅓ by wt. 800-1200 KD NaHA ⅖ by wt, 8-20 KD MW NaHA, and ⅗ by wt. 800-1200 KD NaHA 9/10 by wt, 8-20 KD MW NaHA, and 1/10 by wt. 800-1200 KD NaHA.

HA not only hydrates the skin, but also assists with the proper functioning of the actual cells in the skin and has a structure stabilizing function as well. Low molecular weight hyaluronic acid has been cited as being effective against oxygen free radicals. Trabucchi, E., et al., Low molecular weight hyaluronic acid prevents oxygen free radical damage to granulation tissue during wound healing, Int J Tissue React., 2002; 24(2):65-71.

Hyaluronic acid maintains moisture; decreases inflammation (native macromolecule); stimulates healing (HA fragments); and contributes to proper cell alignment.

### Colloidal Oatmeal

Oatmeal has been used for centuries as a soothing agent to relieve itch and irritation associated with various xerotic dermatoses. In 1945, a ready to use colloidal oatmeal, produced by finely grinding the oat and boiling it to extract the colloidal material, became available. Colloidal oatmeal is available in various dosage forms from powders for the bath to shampoos, shaving gels, and moisturizing creams. Currently, the use of colloidal oatmeal as a skin protectant is regulated by the U.S. Food and Drug Administration (FDA) according to the Over-The-Counter Final Monograph for Skin Protectant Drug Products issued in June 2003. Its preparation is also standardized by the United States Pharmacopeia. The many clinical properties of colloidal oatmeal derive from its chemical polymorphism. The high concentration in starches and beta-glucan is responsible for the protective and water-holding functions of oat. The presence of different types of phenols confers antioxidant and anti-inflammatory activity. Some of the oat phenols are also strong ultraviolet absorbers. The cleansing activity of oat is mostly due to saponins. Its many functional properties make colloidal oatmeal a cleanser, moisturizer, buffer, as well as a soothing and protective anti-inflammatory agent.

Collodial oatmeal can be 0-5% by weight of the compositions, advantageously 0.5-3% by weight.

### Petrolatum

Petroleum jelly, petrolatum, white petrolatum, soft paraffin/paraffin wax or multi-hydrocarbon, CAS number 8009-03-8, is a semi-solid mixture of hydrocarbons (with carbon numbers mainly higher than 25), originally promoted as a topical ointment for its healing properties.

After petroleum jelly became a medicine chest staple, consumers began to use it for many ailments, as well as cosmetic purposes. Its folkloric medicinal value as a “cure-all” has since been limited by better scientific understanding of appropriate and inappropriate uses. It is recognized by the U.S. Food and Drug Administration (FDA) as an approved over-the-counter (OTC) skin protectant and remains widely used in cosmetic skin care.

Petrolatum can be 0-50% by weight of the compositions, advantageously 20-45% or 30-40% by weight.

### Dimethicone

Polydimethylsiloxane (PDMS), also known as dimethylpolysiloxane or dimethicone, belongs to a group of polymeric organosilicon compounds that are commonly referred to as silicones. PDMS is the most widely used silicon-based organic polymer, and is particularly known for its unusual rheological (or flow) properties. PDMS is optically clear, and, in general, inert, non-toxic, and non-flammable. It is one of several types of silicone oil (polymerized siloxane). Its applications range from contact lenses and medical devices to elastomers; it is also present in shampoos (as dimethicone makes hair shiny and slippery), food (antifoaming agent), and lubricants.

Dimethicone can be 0-5% by weight of the compositions, advantageously 1-2% by weight.

### DNA Repair Agents

DNA repair agents such as UV endonuclease and photolyase Synoxyl AZ (Acetyl Zingerone) or Unirepair T-43 (amino acids such as acetyl tyrosine and proline, a hydrolyzed vegetable protein extract and adenosine triphosphate) can be added to the formulations of the disclosure.

### Vehicles and Carriers

The compositions of the present disclosure may be advantageously formulated in a dermatologically acceptable carrier. The carrier used is a carrier suitable for use in topical compositions wherein the active ingredients, which include one or more polyphenols (e.g., flavonoids or flavonoid derivatives), HA and optional additives (e.g., one or more compounds that regulate cell differentiation and/or cell proliferation, one or more antioxidants), are dissolved, dispersed and/or suspended in the composition. The carrier typically includes moisturizing ingredients (that retain moisture and/or provide moisture).

Exemplary formulations include creams, ointments, lotions, pastes, jellies, sprays, shampoos, face soaps, conditioning cleansers, conditioners, aerosols, bath oils, etc. that accomplish direct contact between the active ingredients of the composition and the pores of the skin.

A dermatologically acceptable carrier typically includes ingredients that are chemically and physically compatible with polyphenols/flavonoids, stable with an adequate shelf life, and that aid in penetration of the active ingredient(s) into the skin (e.g., to the epidermis and/or dermis). Optionally, the dermatological carrier contains ingredients that contribute to the ease of application and have pleasing aesthetic properties (color, scent, feel etc.).

The vehicle(s) can include water, ethanol, isopropanol, benzyl alcohol, glycol (e.g., polyethylene glycols, propylene glycol, ethoxydiglycol, and so forth), oils (such as grapeseed, jojoba, coconut, sesame, mineral etc.), glycerol, fatty acid esters, dimethyl isosorbide, surfactants, gelling agents, as well as combinations comprising at least one of the foregoing carriers.

Advantageously, the formulations are greater than 75% by wt. water, or water and petrolatum, or more advantageously, greater than 80% water, or water and petrolatum. The water levels for the lotion is typically 78-85% by wt., and for the serum is 81-88% by wt.

Vehicle components can be chosen to solubilize or disperse colloidal microparticulates of the active ingredients at the desired concentrations, in other words, an acceptable carrier is a vehicle wherein the active ingredients (including the polyphenols/flavonoids and/or hydrated forms) are dissolved and/or dispersed and suspended as microparticulates.

Vehicle components in addition to water and oils can also include liquid emollients, solid emollients, solvents, humectants, thickeners, powders, as well as combinations comprising at least one of the foregoing. Exemplary solvents include ethyl alcohol, isopropanol, ethoxydiglycol, and dimethyl isosorbide, and acetone, as the prevention and/or relief of dryness, and/or for the protection of the skin, such as stearyl alcohol, cetyl alcohol, acetylated lanolin alcohols, stearic acid, isobutyl palmitate, isocetyl stearate, cetyl palmitate, isopropyl stearate, butyl stearate, lanolin, cocoa butter, shea butter, oil (e.g., olive oil, sunflower seed oil, avocado oil, mineral oil), petroleum jelly, and myristate (e.g., butyl myristate, isopropyl myristate, myristyl myristate), as well as combinations comprising at least one of the foregoing.

For skin products, those vehicles that are fat-soluble, can effectively penetrate the stratum corneum and deliver the polyphenol, e.g. flavone(s) to the lipid-rich layers of the skin.

The polyphenol/flavonoid can be loaded into a formulation by adding it into an oil/water (“o/w”) and/or water/oil/water (“w/o/w”) emulsion, which can comprise dispersant(s), emulsifier(s), surfactant(s), solubilizing agent(s) and the like.

It is noted that, while the vehicle for the polyphenol can comprise a relatively simple solvent or dispersant (such as oils and organic alcohols), it is generally preferred that the carrier comprise a composition more conducive to topical application, and particularly one which will form a film or layer on the skin to which it is applied so as to localize the application and provide some resistance to stratum corneum water loss and/or one which aids in delivery to the skin (e.g., to the skin's subsurface layers) and penetration of the active ingredients into the stratum corneum/lipid layers of the skin. Many such compositions take the form of lotions, creams, sprays and gels.

Typical compositions include lotions containing water and/or alcohols, emollients (such as hydrocarbon oils, hydrocarbon waxes, silicone oils, vegetable fats and/or oils, animal fats and/or oils, marine fats and/or oils, glyceride derivatives, fatty acids, fatty acid esters, alcohols (e.g., polyhydric alcohols, alcohol ethers), lanolin (including derivatives), esters (e.g., polyhydric esters, wax esters), sterols, phospholipids, as well as combinations comprising at least one of the foregoing), and generally also emulsifiers (nonionic, cationic or anionic). These same general ingredients can be formulated into a cream rather than a lotion, or into gels, by utilization of different proportions of the ingredients and/or by inclusion of thickening agents such as gums or other forms of hydrophilic colloids.

In one embodiment, the formulation comprises the polyphenols/flavonoids in both the dissolved and dispersed (e.g., microparticulate) forms. The dissolved form(s) can penetrate the skin layers to become bioactive while the dispersed hydrates can serve as a reservoir for maintaining a dissolved concentration level as the dissolved hydrates are consumed so as to maintain sustained flavonoid delivery.

A formulation can be prepared using a lecithin-based oil-in-water cream with about 2.0 wt % apigenin and/or hydrated apigenin and about 0.5 wt % ascorbic acid, with about 0.5 wt % tocotrienol acetate and about 0.25 wt % glycolic acid with the balance comprising the vehicle's components, based upon a total weight of the formulation.

In another example, the formulation can be prepared using a lecithin-based oil in water cream, 3.0 wt % with lecithin, about 0.5 wt % ascorbic acid, about 0.5 wt % tocotrienol acetate, about 0.25 wt % glycolic acid, with the balance comprising the vehicle's components, based upon a total weight of the formulation.

### Additives

The formulation can further comprise additive(s) so long as the specific additive(s) do not adversely affect the active ingredients. Some additive(s) that can be used in the various embodiments of the formulation include:

argan oils, avocado oils
antioxidant(s) (e.g., tocopherol, tocopheryl acetate, butylated hydroxytoluene, sodium metabisulfite, sodium thiosulfate, and propyl gallate),
surfactant(s) (e.g., that can reduce the interfacial tension between phases and/or improve stability of the formulation, and/or that can act as emulsifiers, such as glyceryl stearate, stearyl alcohol, cetyl alcohol, stearic acid dimethicone, a silicone (siloxane) surfactant, polysorbates, sodium laureth),
skin conditioning agent(s) such as silicone oils,
preservative(s) (e.g., methylparaben, propylparaben, benzyl alcohol, benzalkonium chloride etc.),
humectants(s) or emollients or moisturizers such as glycerol, polyethylene glycol, glycerin, sorbitol, mineral oil, isopropyl myristate, etc. Humectants, including glycerin, lecithin, and propylene glycol, draw water into the outer layer of skin. Emollient(s) are substances that soften and moisturize the skin and decrease itching and flaking. Dry skin is caused by a loss of water in the upper layer of the skin. Emollients/moisturizers work by forming an oily layer on the top of the skin that traps water in the skin. Petrolatum, lanolin, and mineral oil are common emollients,
gorgonian extract
buffer(s) (such as phosphate buffers, citrate buffers, and acetate buffers, etc.) pH adjusters such as triethanolamine, potassium hydroxide, sodium hydroxide), hydrochloric acid and phosphoric acid etc.,
gelling agents such as hydroxypropyl ethyl cellulose, hydroxyethyl cellulose, polyacrylic acid polymers, and poloxamers, etc.,
vitamin(s) (e.g., A, B (e.g. B3 and B5) C, D, E, K, etc.), mineral(s), plant extract(s) (e.g., aloe vera, witch hazel, elderflower, cucumber, chamomile, etc.), Vitamins A, C and E are considered powerful antioxidants that fight age-related free radical damage to the skin. Retinol works to increase cell turnover by stimulating cell production underneath the skin, while vitamins C and E remove potentially damaging oxidizing agents.
retinoids
anti-inflammatory agent(s), e.g. corticosteroids
anti-microbial agent(s)
anti-fungal agent(s)
antibacterial agent(s),
moisturizer(s),
skin protectant(s),
silicone(s),
analgesic(s),
anesthetics such as lidocaine
skin penetration enhancer(s), such as propylene glycol, transcutol, isopropyl myristate,
colorant(s) such as yellow no. 5,
fragrance(s) (or perfume),
wax(es) (e.g., beeswax, paraffin wax, etc.),
peptides/amino acids help boost collagen production and provide strength behind skin structure,
ceramides are a type of fat molecule found naturally in the top layer of your skin. Essential to healthy functioning of the skin barrier, ceramides play a key role in helping your skin retain moisture,
aloe acts as a protective layer on the skin and helps replenish its moisture. Aloe vera gel contains two hormones: Auxin and Gibberellins. These two hormones provide wound healing and anti-inflammatory properties that reduce skin inflammation. Giberellin in aloe vera acts as a growth hormone stimulating the growth of new cells. It allows the skin to heal quickly and naturally with minimal scarring. Aloe is soothing and can reduce skin inflammations, blistering and itchiness, while helping the skin to heal more rapidly,
tumeric helps heal and prevent dry skin, and to slow the skin aging process, and is used to diminish wrinkles, keep skin supple and improve skin's elasticity,
ginger stops free radicals that are damaging to our skin and reduces inflammation caused by pimples or other unsightly blemishes. It's also amazing at increasing scalp circulation and stimulating hair growth,
manuka honey has antimicrobial properties,
arnica is used in liniment and ointment preparations used for strains, sprains, and bruises,
lower levels of CoQ10 lead to the skin symptoms associated with aging, including deeper and more pronounced wrinkles. CoQ10 is “highly effective” in protecting skin from such ultraviolet damage. CoQ10 is absorbed into the skin and builds up over time when continually applied.

Optionally, the compositions can further comprise: (i) an additive selected from the group consisting of minerals, plant extracts, concentrates of plant extracts, skin soothing ingredients, colorants, perfumes (fragrances), preservatives, pH adjusters.

Ranges disclosed herein are inclusive and combinable (e.g., ranges of “up to 25 wt. %, or, more specifically, 0.5 wt. % to 5 wt. %”, is inclusive of the endpoints and all intermediate values of the ranges of “5 wt. % to 25 wt. %,” etc.). “Combination” is inclusive of blends, mixtures, alloys, reaction products, and the like. Furthermore, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments. As used herein, the term “(meth)acrylate” encompasses both acrylate and methacrylate groups.

### Advantageous Formulations

Advantageous aqueous formulations include apigenin, HA, Vitamins B3, B5; HA (two weights—high molecular weight, low molecular weight), and have a pH: ˜4-6. See table below.

Table of Advantageous Formulations (% by wt.)

| | | | | |
|---|---|---|---|---|
| Flavonoid, e.g. | 0.01-20 | 0.5-1.5% | 1-5% | 1-5% |
| Apigenin | | | | |
| Hyaluronic Acid | 0-15% | 0.5-3% | 1% | 1-2% |
| Low MW | ⅔ of HA | ⅔ | ⅔ | ⅔ |
| High MW | ⅓ of HA | ⅓ | ⅓ | ⅓ |
| Water | 40-85% | 85% | 45% | 75% |
| Petrolatum | 0-40% | 0-40% | 30% | 0% |
| Colloidal Oatmeal | 0.03-1% | 0-3% | 1% | 0-1% |
| Dimethicone | 0-2.5% | 1% | 0.5-2.5% | 1% |
| Emulsifier | 0-10% | 0-10% | 7% | 2% |
| Humectant, e.g. | 0-2% | 0-2% | 2% | 2% |
| Glycerine | | | | |
| Gorgonian | 0-1% | 0-1% | 0.4% | 1% |
| Extract | | | | |
| Argan | 0-1% | 0-1% | 1% | 1% |
| Avacado Oils | 0-1% | 0-1% | 1% | 1% |
| Vitamins, e.g. | 0-1% | 0-1% | 0.5% | 0.5% |
| B3, B5 | | | | |
| DNA Repair | 0-1% | 0-1% | 1% | 0% |
| Agent | | | | |

### Methods of Preparing Microparticulate Formulations

This disclosure relates to methods of producing a hydrated flavonoid, comprising mixing a polyphenol/flavonoid with an alkali metal hydroxide to form an aqueous solution of an alkali metal flavonoid salt; acidifying the aqueous solution of an alkaline metal flavonoid salt with an acidic agent to a pH level of less than or equal to 7 to form a hydrated flavonoid precipitate, wherein the acidifying step is typically done under conditions producing nanofibers having an average size of 50-1000 nanometers, more advantageously 200-500 nanometers, with an aspect ratio measuring greater than 20. After the acidifying step can be the step of adjusting the pH to less than 7, and filtering the precipitate (to achieve e.g. 10, 20, 25 or 30% apigenin). Alternatively, the precipitate can be dried. The precipitate can then be added to a dermatologically acceptable topical carrier.

Another embodiment is a method of preparing a topical formulation of a (non-hydrated) flavonoid comprising: adding a flavonoid to an emulsion carrier to form a mixture; heating (typically about 120° F.-170° F.) the mixture until it has the approximate viscosity of water (or a viscosity where a dispersion can be done), forming a dispersion of microparticles in the mixture. Typically, the emulsion is an oil in water, or water in oil emulsion and the emulsion includes a stabilizer, a dispersant or a surfactant, or another stabilizing agent to inhibit microparticle settling and agglomeration. In one embodiment, the forming of a dispersion is accomplished using sonication or high pressure homogenization.

### The Preparation of Hydrated Flavonoid Microparticles

Methods are disclosed herein for producing hydrated polyflavonoids of relatively water insoluble flavonoids, such as apigenin and/or luteolin. For example, the hydrated flavones can comprise hydrated apigenin, hydrated luteolin, or a combination thereof, or one of the forgoing hydrated flavones and another flavone or bioflavone. The preparation of these hydrated flavonoids has resulted in the addition of flavonoids to a variety of acceptable pharmaceutical and cosmetic carriers, e.g. aqueous alcoholic solvents with enhanced bioavailability.

In one embodiment, a hydrated flavonoid or flavone is formed by: the mixing of a flavonoid with an aqueous solution and an alkali metal component (e.g., alkali metal hydroxide(s) and/or alkaline metal salt(s)) to form an alkali metal flavonoid salt; adjusting (e.g., acidifying) the alkali metal flavonoid salt mixture with an agent (e.g., an acidic agent) to a pH level of less than or equal to 7.5 resulting in a gel like precipitate of the flavonoid; filtering out the hydrated flavonoid; and washing of the hydrated flavonoid (e.g., with water such as distilled water) to remove soluble alkaline salts and excess acidifying agent; and, optionally, drying of the hydrated flavonoid.

To form fine submicron particles, control of the acidification process is required. This includes the rapid addition and mixing of the acidifying agents with the alkaline flavonoid salt solutions until the microparticulates are uniformly distributed. The mixing of the acidifying agent, at temperatures advantageously from 1 to 10° C., with the alkaline salt solution is done such that the ratio of mixing time to precipitation time is minimized (advantageously, a ratio of 1-5, and most advantageously, a ratio of 1-2). These ratios contribute to increasing the rate of nuclei formation and limit the rate of crystal growth. Typically, the microparticulate hydrated flavonoid has an average size of 50-1000, advantageously 200-500 nanometers, e.g. averaging 250 nm.

Exemplary polyphenols include, for example apigenin, luteolin, curcumin or a combination thereof. Thus, the method can prepare hydrated flavones including hydrated apigenin, hydrated luteolin, or a combination thereof.

The hydrated flavonoids and hydrated flavones are exceptionally beneficial as additives to topical formulations for their anti-cancer, anti-oxidant, anti-inflammatory, UV skin protection and other desirable activities.

### Filtration of the Hydrated Flavonoid Microparticles

The filtration process to separate the hydrated flavonoid precipitate from solution was carried out without the addition of surfactants/dispersants when the pH of the solution is acidified to a pH<7, advantageously <6. Under these conditions, there is entrapment of nearly all nanoparticles (or nanofibers) on a 2 micron filter such that relatively insignificant quantity of nanofibers appeared in the filtrate.

The complete removal of the dispersion medium from nanoparticulates generally requires a separation processes such as centrifuging, lyophilization (freeze-drying), and/or flash spray drying processing. For several applications/formulations, the hydrated flavones/flavonoids precipitates are directly added to formulations without further processing to completely remove the residual water content.

### Sonication

Dispersion and deagglomeration by sonication are a result of ultrasonic cavitation. When exposing liquids to ultrasound, the sound waves that propagate into the liquid result in alternating high-pressure and low-pressure cycles. This applies mechanical stress on the attracting forces between the individual particles. Ultrasonic cavitation in liquids causes high-speed liquid jets of up to 1000 km/hr (approx. 600 mph). Such jets press liquid at high pressure between the particles and separate them from each other. Smaller particles are accelerated with the liquid jets and collide at high speeds. This makes ultrasound an effective means for the dispersing but also for the milling of micron-size and sub-micron-size particles.

Other methods for producing nanoparticles include microprecipitation processes as described in U.S. Pat. Nos. 4,826,689 and 5,314,506; solvent/anti-solvent methods as described in WO 01/92293, 96/32095, 00/44468, 00/38811; and melt emulsification processes as described in WO98/32095 & 99/59709.

The Preparation of Flavonoid Microparticulates Via In-Situ Methods with the Emulsified Carrier

Previous practices required that submicron particulates first be formed via a variety of processing methods which include mechanical (pearl milling, high pressure homogenization (HPH)), precipitation etc. Further, time-consuming and costly filtration, evaporative techniques (flash spray drying, freeze drying etc.) are required to separate the liquid medium from the submicron size particulates prior to inclusion within topical formulation.

In another embodiment, unprocessed apigenin powder or another relatively insoluble flavonoid, is directly added to an oil in water, or water in oil emulsion, and processed via sonication and/or HPH techniques to achieve a dispersion of microparticulates. A requirement of the method is that sonication and/or HPH processing of the emulsions be carried out at elevated temperatures such that the viscosity of the fluid mixture is reduced to approximately viscosity levels of water. Fine submicron particulates are formed when the fluid mixtures are sonicated at temperatures of about 120° F.-170° F. Further, the stabilizing additives of the emulsion such as dispersants, surfactants and other stabilizing agents serve to inhibit further potential particulate agglomeration. This in-situ processing methodology is a cost effective and less time consuming processing method to achieve submicron sized particulates within formulations, including topical formulations.

### Methods of Treatment

Embodiments of the present disclosure relate to methods of prevention and treatment of radiation dermatitis, thermal burns, wounds and cancers.

### Radiation Dermatitis

A radiation burn is damage to the skin or other biological tissue caused by exposure to radiation. Of great concern is ionizing radiation. High exposure to X-rays during diagnostic medical imaging or radiotherapy can also result in radiation burns. As the ionizing radiation interacts with cells within the body—damaging them—the body responds to this damage, typically resulting in erythema—that is, redness around the damaged area.

### External-Beam Radiation Therapy

External-beam radiation therapy is most often delivered in the form of photon beams (either x-rays or gamma rays). A photon is the basic unit of light and other forms of electromagnetic radiation. It can be thought of as a bundle of energy. The amount of energy in a photon can vary. For example, the photons in gamma rays have the highest energy, followed by the photons in x-rays.

Linear Accelerator Used for External-beam Radiation Therapy. Many types of external-beam radiation therapy are delivered using a machine called a linear accelerator (also called a LINAC). A LINAC uses electricity to form a stream of fast-moving subatomic particles. This creates high-energy radiation that may be used to treat cancer.

Many types of external-beam radiation therapy are delivered using a machine called a linear accelerator (also called a LINAC). A LINAC uses electricity to form a stream of fast-moving subatomic particles. This creates high-energy radiation that can be used to treat cancer.

Patients usually receive external-beam radiation therapy in daily treatment sessions over the course of several weeks. The number of treatment sessions depends on many factors, including the total radiation dose that will be given.

One of the most common types of external-beam radiation therapy is called 3-dimensional conformal radiation therapy (3D-CRT). 3D-CRT uses sophisticated computer software and advanced treatment machines to deliver radiation to precisely shaped target areas.

For patients having head and neck cancer (with a mucosal primary site including the nasopharynx, oropharynx, oral cavity, larynx and hypopharynx) radiotherapy is typically scheduled. For radiation indicated to bilateral sides of the necks, daily radiation fraction dose of 1.6-2.1 Gray (Gy) and ≥44 Gy is delivered comprehensively to both sides of the necks utilizing intensity modulated or three-dimensional conformal treatment delivery techniques. The treatment consists of radiotherapy (usually 6 to 7 weeks, depending on the number of daily radiotherapy treatments required). As many as 95% of patients treated with radiation therapy for cancer will experience a skin reaction. Some reactions are immediate, while others are later (e.g., months after treatment).

Radiation dermatitis (also known as radiodermatitis) is caused by the changes cells undergo in the basal layer of the epidermis and dermis. Cumulative daily doses of radiation to the treatment field prevent normal skin cells from being repopulated from the basal layer which weakens skin integrity. The effects of radiation dermatitis can impact quality of life, cause pain and discomfort, limit activities and delay treatment.

Radiodermatitis can be acute or chronic, and includes localized erythema and edema, skin shedding (desquamation), hair loss (epilation), fibrosis, and necrosis.

Erythema and swelling may begin within hours or days of initiating radiation therapy due to the release of cytokines that cause capillary dilation, leukocyte infiltration, and localized swelling.

Dryness and epilation may occur within days to weeks due to damage of sebaceous glands and hair follicles respectively in the dermal layer.

Dry desquamation, characterized by dryness, scaling, and pruritus, typically can occur after the third week or after a cumulative dose of 30 Gy due to destruction of regenerative basal cells. Dry desquamation typically resolves within one to two weeks of therapy.

Moist desquamation, evidenced by red, exposed dermis and serous oozing, occurs after four to five weeks of therapy or with 45 to 60 Gy cumulative dose as the basal cells are further depleted.

### Factors Contributing to Response

Fraction size, type of energy and use of bolus doses; prolonged or multiple procedures requiring radiation exposure; total radiation doses of greater than 55 Gy, or large individual doses per fraction (greater than 3-4 Gy per dose) all contribute to the patient response.

### Common Post Radiation Treatment Side Effects

Changes in skin may appear several months to years after radiation therapy has been completed. These changes in pigmentation are caused by the damaging effects of radiation on melanocytes. Telangectasia results from damage and stretching of capillaries commonly found with moist desquamation. Fibrosis caused by excessive extracellular matrix and collagen deposits because of inflammatory response which can lead to decreased tissue flexibility causing reduced range of motion, atrophy and reduced tissue strength. Increased risk for delayed wound healing, dehiscence, fistula, tissue graft failures and other surgical complications.

Radiation dermatitis occurs to some degree in most patients receiving radiation therapy, with or without chemotherapy. There are three specific types of radiodermatitis: i) acute radiodermatitis, ii) chronic radiodermatitis, and iii) eosinophilic, polymorphic, and pruritic eruption associated with radiotherapy.

With interventional fluoroscopy, because of the high skin doses that can be generated in the course of the intervention, some procedures have resulted in early (less than two months after exposure) and/or late (two months or more after exposure) skin reactions, including necrosis in some cases.

Radiation dermatitis, in the form of intense erythema and vesiculation of the skin, may be observed in radiation ports.

Acute radiodermatitis occurs when an “erythema dose” of ionizing radiation is given to the skin, after which visible erythema appears up to 24 hours after. Radiation dermatitis generally manifests within a few weeks after the start of radiotherapy. Acute radiodermatitis, while presenting as red patches, may sometimes also present with desquamation or blistering. Erythema may occur at a dose of 2 Gy radiation or greater. Acute radiodermatitis usually resolves within three to four weeks after therapy.

Acute radiation dermatitis occurs within 90 days of exposure to radiation. Patient may have skin changes ranging from faint erythema (reddening) and desquamation (peeling skin) to skin necrosis (death of skin cells) and ulceration. The National Cancer Institute has developed a 4 stage criteria for the classification of acute radiation dermatitis:
Grade 1—Faint erythema or desquamation.
   Characterized by mild, blanchable, erythema or dry desquamation.
   Onset is typically within days to weeks of initiating therapy and symptoms may fade within a month.
   Dry desquamation may be associated with pruritus, epilation, scaling, and possibly changes in pigmentation.
   Patients with mild radiodermatitis may report that their skin feels tight.
   Hair loss occurs in the treatment field and is often temporary, but may become permanent in some patients.
Grade 2—Moderate to brisk erythema or patchy, moist desquamation confined to skin folds and creases. Moderate swelling.
   Often painful and presents as edema and moist desquamation that is localized to the skin folds.
   Bullae (fluid filled sacs or lesions) may also be present.
   Wet desquamation indicates that the integrity of the dermis is impaired and thus patients are at increased risk for infection with Staphylococcus aureus.
   Patients with wet desquamation should be monitored for evidence of infection, such as purulent drainage and fevers, and educated on the need to report any symptoms of infection.
Grade 3—Confluent, moist desquamation greater than 1.5 cm diameter, which is not confined to the skin folds. Pitting edema (severe swelling).
   The area of moist desquamation has spread to areas outside of the skin folds
Grade 4—Skin necrosis or ulceration of full thickness dermis (middle layer of skin).
   Ulcers, hemorrhages, and/or tissue necrosis are present.
   Ulcers may be red with raised edges with a red or black base (McQuestion, 2006).
   Unfortunately, ulceration is very painful and does not heal well (Gerlach, 2005).

Chronic radiodermatitis occurs with chronic exposure to “sub-erythema” doses of ionizing radiation over a prolonged period, producing varying degrees of damage to the skin and its underlying parts after a variable latent period of several months to several decades. Historically, this type of radiation reaction occurred most frequently in radiologists and radiographers who were constantly exposed to ionizing radiation, especially before the use of x-ray filters. Chronic radiodermatitis, squamous and basal cell carcinomas can develop months to years after radiation exposure. Clinically, chronic radiodermatitis presents as atrophic indurated plaques, often whitish or yellowish, with telangiectasia, sometimes with hyperkeratosis.

Chronic radiation dermatitis may occur from days 15 to 10 years or more after the beginning of radiation therapy. It is an extension of the acute process and involves further inflammatory changes of the skin. Chronic radiation-induced changes in the skin are characterized by: disappearance of follicular structures (pores), increase in collagen and damage to elastic fibers in the dermis, fragile surface skin (epidermis), and telangiectasia (prominent blood vessels).

Eosinophilic, polymorphic, and pruritic eruption associated with radiotherapy is a skin condition that occurs most often in women receiving cobalt radiotherapy for internal cancer.

### Delayed Effects

Radiation acne is a cutaneous condition characterized by comedo-like papules occurring at sites of previous exposure to therapeutic ionizing radiation—skin lesions that begin to appear as the acute phase of radiation dermatitis begins to resolve.

Radiation recall reactions occur months to years after radiation treatment, a reaction that follows recent administration of a chemotherapeutic agent and occurs with the prior radiation port, characterized by features of radiation dermatitis. Radiation recall dermatitis is an inflammatory skin reaction that occurs in a previously irradiated body part following drug administration. There does not appear to be a minimum dose, nor an established radiotherapy dose relationship.

This disclosure relates to methods of treating skin damaged by ionizing radiation exposure including radiation dermatitis comprising topically applying to the damaged skin of a mammal having received ionizing radiation, a therapeutically effective amount of a formulation comprising a hydrated or solubilized polyphenol (e.g. flavonoid), HA, and a carrier that permits delivery of the flavonoid to the stratus corneum, the epidermis and dermis of the skin.

This disclosure also relates to methods of preventing the damaging effects to the skin of ionizing radiation exposure including radiation dermatitis comprising topically applying to the skin of a mammal about to receive ionizing radiation (1-2 week prior to radiation but stopping administration at least 3 hours prior to radiotherapy), a therapeutically effective amount of a formulation comprising a hydrated or solubilized polyphenol (e.g. flavonoid), HA, and a carrier that permits delivery of the flavonoid to the stratus corneum, the epidermis and dermis of the skin. During the period of radiotherapy treatment, the formulation can be applied 1, 2 or 3 times daily every day that there is no radiotherapy.

Treatment of Tumors with Radiation and Polyphenols

Embodiments may also relate to methods of treating cancer in a mammal having a tumor comprising:

administering a therapeutically effective dose of radiation to said tumor,
applying to the skin of the mammal receiving the radiation, a therapeutically effective amount of a formulation comprising a hydrated or solubilized polyphenol, and a carrier that permits delivery of the polyphenol to the stratus corneum and the epidermis and the dermis of the skin. During the period of radiotherapy, the formulation is typically applied daily except on days that there is radiotherapy.

### Thermal Burns

The formulations been found be effective in ameliorating skin burns by accelerating the healing processes, and reducing pain and discomfort. The formulations are an effective remedy for minor skin burns (primarily 1^{st} and 2^{nd} degree). The formulations have demonstrated rapid healing with relatively no scarring effects for minor and 2^{nd} degree burns. See Examples below.

Embodiments may also relate to methods of preventing skin infections and minimizing scars while accelerating the healing of burns.

Thermal burns can result in a loss of a large area of skin and the resulting scar will contract, causing the edges of skin to be pulled together, affecting adjacent muscles and tendons and restricting normal movement. These are usually treated with scar removal surgery using a skin flap, or graft and tissue expansion but can also be treated now with a biological skin revival cream.

A thermal burn can also yield abnormal scarring or scars that go beyond the site of injury called keloid scars or it may result in excessively fibrotic scars called hypertrophic scars. Burn-related skin fibrosis leads to loss of tissue function and hypertrophic scar formation with damaging consequences for the burned person. Burn and wound treatment includes keeping the burn clean to prevent infection.

Thermal burns are classified as first, second or third degree burns depending on the amount and depth of tissue damage.

1st Degree Burn is a superficial, reddened area of skin like sunburn. A first-degree burn causes damage to the epidermis, causing pain, redness and some swelling. Typically, this type of burn will heal without scarring.
2nd Degree Burn is a blistered injury site which may heal spontaneously after the blister fluid has been removed. A second-degree burn causes damage to the epidermis and the dermis, and this burn usually results in pain, redness and blistering.
3rd Degree Burn is a burn through the entire skin and will usually require surgical intervention for wound healing. Third degree burns are the most severe because the damage extends past the upper layers of skin to the sensitive subcutaneous tissue, destroying nerves, blood vessels, and other dermal components. Extensive third degree burns can be fatal because the threat of infection is extremely high. In fact, bacterial infection is the leading cause of death in burn victims.

Because of the availability of several effective topical agents, wound care protocols may vary and still meet with success. Observation of the wound, along with the appropriate selection of a topical therapeutic agent, can improve the healing of wound.

Embodiments relate to methods of treating skin damaged by thermal burn comprising topically applying to the damaged skin of a mammal, a therapeutically effective amount of a formulation comprising a hydrated or solubilized polyphenol (e.g. flavonoid), HA, and a carrier that permits delivery of the flavonoid to the stratus corneum, the epidermis and dermis of the skin. Typically a formulation is applied 1, 2 or 3 times daily until the damaged skin has healed.

### Wounds

The disclosure also relates to methods of preventing skin infections and minimizing scars while accelerating the healing of wounds, including cuts and scrapes. Typically a formulation of the present disclosure is applied 1, 2 or 3 times daily until the damaged skin has healed.

### Non Melanoma Skin Cancers and Xeroderma Pigmentosum

Nonmelanoma skin cancer refers to all the types of cancer that occur in the skin that are not melanoma.

Several types of skin cancer fall within the broader category of nonmelanoma skin cancer, with the most common types being basal cell carcinoma and squamous cell carcinoma.

Nonmelanoma skin cancer includes:
Basal cell carcinoma
Squamous cell carcinoma
Angiosarcoma
Cutaneous B-cell lymphoma
Cutaneous T-cell lymphoma
Dermatofibrosarcoma protuberans
Merkel cell carcinoma
Sebaceous gland carcinoma
Cancer that spreads from other areas of the body to the skin, such as breast cancer and mouth cancer

The drug most often used in topical treatment of actinic keratoses, as well as some basal and squamous cell skin cancers, is 5-fluorouracil, or 5-FU (Efudex®, Carac®, Fluoroplex®, others). It is typically applied to the skin once or twice a day for several weeks. When applied directly on the skin, 5-FU kills tumor cells near the skin's surface.

This disclosure relates to methods of treating nonmelanoma skin cancer comprising topically applying to the damaged skin of a mammal, a therapeutically effective amount of a formulation comprising 5-FU, a hydrated or solubilized polyphenol (e.g. flavonoid), HA, and a carrier. Advantageously, the formulation of the disclosure is applied 1, 2 or 3 times daily.

### Xeroderma Pigmentosum

Xeroderma pigmentosum, which is commonly known as XP, is an inherited condition characterized by an extreme sensitivity to ultraviolet (UV) rays from sunlight. UV radiation is very similar in nature to the gamma rays commonly used in radiotherapy, with the major difference being wavelength. This condition mostly affects the eyes and areas of skin exposed to the sun. This disclosure relates to methods of treating xeroderma pigmentosum in a patient comprising topically applying to the damaged skin of said patient, a therapeutically effective amount of a formulation comprising, a hydrated or solubilized polyphenol (e.g. flavonoid), HA, and a carrier. In another embodiment, 5-FU is added to the formulation. Advantageously, the formulation of the disclosure is applied 1, 2 or 3 times daily.

### Eczema

The formulations of this disclosure can be used for the treatment of eczema, e.g. atopic dermatitis. This disclosure relates to methods of treating eczema in a patient comprising topically applying to the damaged skin of said patient, a therapeutically effective amount of a formulation of this disclosure. Types of eczema treatable by the methods of the disclosure include atopic dermatitis, stasis dermatitis, scabies, fungal dermatitis, pompholyx, nummular eczema, lichen simplex chronicus, seborrheic dermatitis, xerotic eczema, and allergic contact dermatitis.

Typically, a formulation of the disclosure is applied 1, 2 or 3 times daily until the damaged skin has healed. In advantageous formulations, diphenyl hydramine hydrochloride or another antihistamine is added, or a hydrocortisone.

### Sunburn

Sunburn is damage to the skin produced by UV rays in sunlight. UV radiation is very similar in nature to the gamma rays commonly used in radiotherapy, with the major difference being wavelength. Advantageously, a formulation of the disclosure is applied prior to, or soon after exposure to sunlight and the noticing of possible sunburn. The formulations can be combined with SPF sunscreens. Advantageously, the formulation is applied 1, 2 or 3 times daily.

### Additional Uses

The formulations of the disclosure have therapeutic value in treating a wide variety of skin problems: psoriasis, rashes, cracked skin, dry skin, keloids, surgical scars and stretch marks. The formulations are useful for treatment of abrasions, itching, athlete's foot, boils, insect bites/stings, poison ivy, oak or sumac, seborrheic dermatitis, skin after chemical (CTCA) peels, dandruff, skin allergies and skin ulcers, and skin after laser treatment of sun damaged skin. See the uses including psoriasis, noted in U.S. Pat. No. 9,889,098 which is hereby incorporated by reference in its entirety.

### Application of the Formulations

Typically, subjects apply the formulations of the disclosure to the affected site one to three times daily. The formulation is evenly spread across the entire affected area. Dosage is typically 1-20% by wt. polyphenol within the formulation. Packaging can be a pump bottle or other packaging that protects product from degradation. Nitrogen can be used to reduce or replace oxygen to increase long term stability of the formulation.

In the case of neck cancer, medication is applied to the entire area of the neck: from the mid-face above the mandible to below the clavicle and from the posterior neck to the anterior midline of the neck at the level of the mid-thyroid cartilage. Starting within 0 to 3 days of radiotherapy, the medication is applied three times daily: after radiation treatment, after showering, and before bedtime. The medication is applied no less than 4 hours before radiotherapy. The study medication is typically applied three times per day for 4 weeks after completion of radiotherapy. At the time of radiation treatment, the skin should be clean, including devoid of any the medication or lotions.

The present invention will be further understood after careful consideration is given to the following non-limiting examples thereof.

### EXAMPLES

### Example 1

### Solubility of Apigenin and Hydrated Apigenin

A good solubility of polyphenols in the alkaline medium is very important in generating well hydrated polyphenols upon acidification. The regenerated hydrated products mix well in the formulations. Sodium hydroxide solution is an effective solubilizer at different concentrations for apigenin, a polyphenol flavonoid. This is exemplified in the table below—

### Apigenin Solubility in NaOH Solutions

| NaOH Molarity | Apigenin Solubility |
|---|---|
| (moles/liter) | (mg/ml) |
| 0.75 | >150 |
| 1.015 | >200 |
| 2.0 | >300 |

Potassium hydroxide (KOH) and lithium hydroxide (LiOH) also solubilize similar quantities of the pale yellow apigenin powder as NaOH solutions.

To form the hydrated apigenin, apigenin powders were initially dissolved in solutions of alkaline metal hydroxides. The hydrated apigenin was formed upon slow acidification of the alkaline apigenin solution (contained in water at a pH of greater than 8) with an acid such as citric acid or hydrochloric acid (HCl) to generate snow-like colloidal (gel-like) precipitate. The precipitation was complete at pH between 4-5. The gel like precipitate was filtered and thoroughly washed with distilled water to remove soluble components. The precipitate was pressed and further exposed to an airflow to further dry the hydrated apigenin product.

Hydrated luteolin (polyphenol flavonoid) and hydrated curcumin (a polyphenol) was formed in a procedure similar to that described for producing hydrated apigenin.

Scanning Electron Microscopy (SEM) images of the hydrated apigenin, and unprocessed apigenin powder were collected to determine particle morphology details. The morphology exhibited by the unprocessed samples are very different than the morphology exhibited by the typical hydrated apigenin samples. The chemical composition as determined by FTIR and Raman Spectroscopy, could not detect any chemical differences in the unprocessed apigenin and the hydrated apigenin. These observations suggested that during the formation of hydrated flavonoids and polyphenols, there has been a change in the crystal shape and/or crystal habit of the flavones, possibly resulting in a polymorph of the flavones. The hydrated apigenin was composed of fibers with diameters of 30-500 nm (a range of being nano-fibers) with aspect ratios measuring greater than 20.

### Example 2

### Production of Hydrated Apigenin Nanoparticles

10 grams of apigenin powder was mixed with 8.8 grams 50% aqueous NaOH and 200 mL deionized water, and stirred under nitrogen until the apigenin was fully dissolved yielding a dark yellow brown solution. With a gentle nitrogen flow and moderate stirring (200-300 rpm), 42.6 grams of 50% aqueous citric acid solution was added slowly to the above alkaline solution. Stirring speed was increased to 400-500 rpm during the acid addition, as mixture thickened and turned grey and opaque. Vigorous stirring was continued until homogenous precipitate was obtained. Precipitated hydrated apigenin solids were filtered and washed repeatedly with deionized water until the wash water pH was between 4.0 and 5.0. The hydrated apigenin was 70-90% water wet. The wet hydrated apigenin was directly used in the formulations. SEM images have shown the hydrated apigenin to be of nano-fiber nature.

### Example 3

### Production of Hydrated Curcumin Nanoparticles

Hydrated curcumin was produced in a similar way as hydrated apigenin of Example 2.

10 grams of yellow curcumin powder was mixed with 2.2 grams 50% aqueous NaOH and 200 mL deionized water, and stirred under nitrogen until the apigenin was fully dissolved yielding a dark yellow red solution. With a gentle nitrogen flow and moderate stirring (200-300 rpm), 10.4 grams of 50% aqueous citric acid solution was added slowly to the above alkaline solution. Stirring speed was increased to 400-500 rpm during the acid addition, as mixture thickened and turned orange. Vigorous stirring was continued until homogenous yellow precipitate was obtained. Precipitated hydrated curcumin solids were filtered and washed repeatedly with deionized water until the wash water pH was between 5 and 6. The hydrated curcumin was 70-90% water wet.

The wet hydrated curcumin can be directly used in the formulations.

### Example 4

### Production of Hyaluronic Acid Gel

Hyaluronic Acid (HA) is an essential component of the formulations. HA, in the form of sodium salt (NaHA), is added in desired quantities to the formulation as a gel, prepared by solubilizing in water. The gel is prepared separately as HA is slow to form a gel solution due to large molecular weight.

67 gm NaHA (8-20 KD MW), and 33 gm NaHA (800-1,200 KD MW) were suspended in 900 gm of cold (4-5° C.) deionized water. The suspension was vigorously stirred with overhead mechanical stirrer over several hours (4-5 hrs). During this period a very thick and difficult to stir gel was formed. This gel was allowed to sit overnight at ambient temperatures. The thick 10% HA gel, thus obtained, was stored for further use in the formulations.

### Example 5

Production of Topical Formulations with Hydrated Nano-Apigenin

It should be understood that various minor changes might be made relating to the manufacturing process conditions without significant alterations in the final apigenin containing product properties.

A 100 gm batch containing 1.5 wt % apigenin and 1 wt % hyaluronic acid in Cetaphil, whose ingredients are listed below, was prepared as follows:

### Cetaphil Ingredients

Water (Purified), Glycerin, Hydrogenated Polyisobutene, Cetearyl Alcohol, Ceteareth 20, Macadamia
Ternifolia (Nut) Oil, Dimethicone, Tocopheryl Acetate (Vitamin E), Stearoxytrimethylsilane, Stearyl
Alcohol, Panthenol, Farnesol, Benzyl Alcohol, Phenoxyethanol, Acrylates/C10 30 Alkyl Acrylate
Crosspolymer, Sodium Hydroxide, Citric Acid

69.6 gm of Cetaphil lotion, 0.4 gm Vitamin B3 (Niacin), and 5 gm deionized water were taken in a 500 mL beaker equipped with an overhead mechanical stirrer. The mixture was warmed to 40° C. while stirring. 15 gm hydrated apigenin (90% water), from Example 2, was added in small portions to the above warm lotion mixture while stirring continuously. After complete addition of hydrated apigenin, heat was removed and the mixture was allowed to cool to ambient temperatures with stirring. 10 gm of hyaluronic acid gel, (10 wt %) from Example 4, was added to the above mixture and stirred vigorously for 1 hr. pH of this lotion was measured to 4.25-5.0. The resulting 100 gm lotion was further mixed for 1 minute with ultrasonic liquid processor Qsonica. Digital Sonicator “Sonicator 4000” providing additional mixing, dispersing and degassing of the mixture. This homogenized lotion was stored in airless dispensing containment tubes for further testing.

Lotions with higher apigenin content were made as indicated in the following table:

Lotions with Variable % of Hydrated Apigenin

| Ingredients | % Apigenin | | | |
|---|---|---|---|---|
| Base Lotion | 69.6 | 69.6 | 69.6 | 69.6 |
| Water | 5 | 10 | 8 | 15 |
| Vitamin B3 | 0.4 | 0.4 | 0.4 | 0.4 |
| Hydrated Nano- | * 15 (1.5%) | * 10(2.5%) | * 12(3%) | * 20 (5%) |
| Apigenin | | | | |
| HA Gel (10 wt %) | 10 | 10 | 10 | 10 |

* Effective Apigenin % from 90% Wet Hydrated Apigenin in Parenthesis

Higher apigenin content hydrated nano-apigenin was difficult to produce since a very thick lotion resulted from the mixing.

### Example 6

### Production of Topical Formulations with Unprocessed Apigenin

A 100 gm batch containing 1.5 wt % apigenin and 1 wt % hyaluronic acid in Cetaphil whose ingredients are listed in Example 5,

69.6 gm of Cetaphil, 0.4 gm Vitamin B3 (Niacin), and 18.5 gm deionized water were taken in a 500 mL beaker equipped with an overhead mechanical stirrer. The mixture was warmed to 40° C. while stirring. 1.5 gm of unprocessed apigenin powder obtained from Skyherb Technologies Co, Hangzhou, China, was added in small portions to the above warm lotion mixture while stirring continuously. After complete addition apigenin, heat was removed and the mixture was allowed to cool to ambient temperatures with stirring. 10 gm of hyaluronic acid gel, (10 wt %) from Example 3, was added to the above mixture and stirred vigorously for 1 hr. The pH of this lotion was adjusted to 4.25-5.0 using 50% citric acid solution.

The resulting 100 gm lotion was further mixed for 1 minute with ultrasonic liquid processor Qsonica Digital. Sonicator “Sonicator 4000” providing additional mixing, dispersing and degassing of the mixture. This homogenized lotion was stored in airless dispensing containment tubes for further testing

This method allows to produce lotion with varying apigenin content by weight percentages as shown in the table below—

Lotion with Variable Apigenin %

| Ingredients | % Apigenin | | | | | | | |
|---|---|---|---|---|---|---|---|---|
| Base Lotion | 69.6 | 69.6 | 69.6 | 69.6 | 69.6 | 69.6 | 69.6 | 69.6 |
| Water | 19.5 | 18.5 | 17.5 | 15 | 12.5 | 10 | 7.5 | 5 |
| Vitamin B3 | 0.4 | 0.4 | 0.4 | 0.4 | 0.4 | 0.4 | 0.4 | 0.4 |
| Apigenin | 0.5 | 1.5 | 2.5 | 5 | 7.5 | 10 | 12.5 | 15 |
| Powder | | | | | | | | |
| HA Gel | 10 | 10 | 10 | 10 | 10 | 10 | 10 | 10 |
| (10 wt %) | | | | | | | | |

The lotion with 10% or more apigenin could be produced, however they were very thick.

Lotions with variable hyaluronic acid (HA) content were also produced. However, there was a limitation as to how high HA content could be used. HA is added to the lotion as a gel, and it was practically very difficult to produce a gel of more than 10% concentration. Lotions were produced up to 5% effective HA concentration as shown in the table below:

Lotions with Variable HA Content

| Ingredients | * % Hyaluronic Acid | | | | | | | |
|---|---|---|---|---|---|---|---|---|
| Base Lotion | 69.6 | 69.6 | 69.6 | 69.6 | 69.6 | 58.6 | 53.6 | 48.6 |
| Water | 29 | 19 | 14 | 11.5 | 9 | 0 | 0 | 0 |
| Vitamin B3 | 0.4 | 0.4 | 0.4 | 0.4 | 0.4 | 0.4 | 0.4 | 0.4 |
| Apigenin Powder | 1 | 1 | 1 | 1 | 1 | 1 | 1 | 1 |
| HA Gel (10 wt %) | 0 | 10(1%) | 15(1.5%) | 17.5(2.5%) | 20(2%) | 40(4%) | 45(4.5) | 50(5.0%) |

The effective % HA is shown in the parentheses in the columns.

### Example 7

Production of Topical Formulations with In-Situ Generated Nano—Apigenin in Lotion

In this Example, apigenin nano-particles were generated in the acid carrying lotion through the acidification of alkaline apigenin sodium or potassium salt solution. The in-situ acidification of apigenin sodium results in a uniform emulsion environment—uniform nano-sized hydrated apigenin well distributed in the lotion. A lotion can be produced in a desirable and controlled concentrations starting with desirable concentrations of apigenin salt solution.
A) A 100 gm batch containing 1.5 wt % apigenin and 1 wt % hyaluronic acid within a Base Moisturizing Lotion (Cetaphil), was prepared as shown below using apigenin—sodium salt

Alkaline apigenin—sodium salt was prepared by mixing 1.5 g apigenin powder with 29 gm solution made with 0.5 gm sodium hydroxide (NaOH) and 28.5 g water. Resulting solution was stirred for an additional 15 min to complete the salt formation. Separately, 54.1 g Base Moisturizing Lotion was mixed and stirred for 15 minutes with 0.4 gm of Niacin (Vitamin B3) and 5.0 gm of 50% citric acid solution. pH of this homogeneous lotion mixture was measured to be between 3.8-4.0. The apigenin—sodium salt solution (made above) was added slowly over 15 minutes to this acidic lotion with additional 15 minutes vigorous stirring. A light cream colored lotion was obtained which had a pH of 4.5 to 4.6. 10 gm (10 wt %) hyaluronic acid gel was added to this lotion and stirred for 30 minutes to obtain a homogeneous cream colored lotion that had a pH of 4.5-4.6.

The method described in this Example allows to conveniently make variable concentration of hydrated nano—Apigenin within the Lotion by choosing defined concentrations of apigenin—sodium salt solution.
B) A 100 gm batch containing 1.5 wt % apigenin and 1 wt % hyaluronic acid within a Base Moisturizing Lotion (Cetaphil), was prepared as shown below using apigenin potassium salt

Alkaline apigenin—podium salt was prepared by mixing 1.5 g apigenin powder with 29 gm solution made with 0.7 gm sodium hydroxide (NaOH) and 28.5 g water. Resulting solution was continued to stir for additional 15 min to complete the salt formation.

Separately, 54.1 g Base Moisturizing Lotion was mixed and stirred for 15 minutes with 0.4 gm of Niacin (Vitamin B3) and 5.0 gm of 50% citric acid solution. The pH of this homogeneous lotion mixture was measured to be between 3.8 and 4.0. The apigenin—sodium salt solution (made above) was added slowly over 15 minutes to this acidic lotion with additional 15 minutes vigorous stirring. A light cream colored lotion was obtained which had a pH of 4.5 to 4.6. 10 gm (10 wt %) hyaluronic acid gel was added to this lotion and stirred for 30 minutes to obtain a homogeneous cream colored lotion that had a pH of 4.5-4.6.

The method described in this Example allows variable concentrations of hydrated nano—apigenin in the lotion by choosing defined concentrations of apigenin—sodium salt solution.

### Example 8

### Production of Base Moisturizing Lotion

The modified base moisturizing lotion was produced in house and the ingredients are listed in the table below:

| | | | % by |
|---|---|---|---|
| Phase | Ingredient | Function | Weight |
| A | Water | Diluent | 87.31 |
| A | Disodium EDTA | Chelating | 0.1 |
| | | Agent | |
| A | Glycerin | Humectant | 3 |
| A | Acrylates/C10 30 Alkyl Acrylate | Thickener | 0.6 |
| | Crosspolymer(Carbopol) | | |
| A | Benzyl Alcohol | Preservative | 0.5 |
| A | Phenoxy Ethanol | Preservative | 0.8 |
| B | Hydrogenated Polyisobutene (Panalene) | Emolient | 2.6 |
| B | Stearoxy Trimethylsilane Steary Alcohol | Emulsifier | 0.52 |
| B | Dimethicone | Emolient | 0.78 |
| B | Macedonia Ternifolia Nut Oil | Emolient | 0.52 |
| B | Cetearfyl Alcohol Ceteareth - 20 | Emulsifier | 1.04 |
| B | Stearyl Alcohol | Emulsifier | 0.52 |
| B | Tocopheryl Acetate | Antioxidant | 0.05 |
| B | Panthenol | Pro-Vitamin | 0.4 |
| | | B5 | |
| C | Sodium Hydroxide (20%) | pH Adjuster | 1.26 |

Phase A ingredients (from the table above) were mixed and heated to 70-75° C. The mixture was maintained at this temperature with vigorous stirring for 15 minutes. The Phase B ingredients were mixed separately and heated to 70-75° C. to obtain a uniform mixture. This was then added to the uniform Phase A. The combined mixture of A & B were continued to stir at 70-75° C. till a homogeneous mixture was obtained. This was cooled to about 60° C. The pH was adjusted to about 7 by slow addition of 20% NaOH (Phase C) while stirring vigorously. Once the lotion was uniform, the heat was discontinued and the lotion was allowed to cool to ambient temperatures. The lotion was stored at room temperature for further use. The lotion is referred to as “Viz base” herein.

### Example 9

### Production of Topical Formulations with Hydrated Nano-Apigenin Using Base Moisturizing Lotion of Example 8

A 100 gm batch containing 1.5 wt % apigenin and 1 wt % hyaluronic acid within the base lotion was prepared as follows:

69.6 gm of Base Moisturizing Lotion, 0.4 gm Vitamin B3 (Niacin), and 5 gm deionized water were taken in a 500 mL beaker equipped with an overhead mechanical stirrer. The mixture was warmed to 40° C. while stirring. 15 gm hydrated apigenin (90% water), from Example 2, was added in small portions to the above warm Lotion mixture while stirring continuously. After complete addition of hydrated apigenin, heat was removed and the mixture was allowed to cool to ambient temperatures with stirring. 10 gm of hyaluronic acid gel, (10 wt %) from Example 4, was added to the above mixture and stirred vigorously for 1 hr. pH of this lotion was measured to 4.25-5.0. The resulting 100 gm lotion was further mixed for 1 minute with ultrasonic liquid processor Qsonica Digital Sonicator “Sonicator 4000” providing additional mixing, dispersing and degassing of the mixture.

This lotion was visually and physically comparable to the lotion from Example 5. It is referred to as “PMC” herein.

### Example 10

### Radiation Dermatitis Study

Twenty-eight subjects were enrolled in a study comparing topical treatment formulations for efficacy in the treatment of radiation dermatitis. The sample size of 28 evaluable subjects is based upon the expectation that data generated from this number of subjects will adequately determine subject receptivity to study medication and generate an effect size estimate for a subsequent larger study.

Subjects were enrolled in the study once they had radiation dermatitis of a severity of at least 2 according to the Radiation Dermatitis Severity Score (RDS) (defined below). Determination of the severity of dermatitis was evaluated using the RDS (adapted from the Radiation Treatment Oncology Group, RTOG, and the National Cancer Institute Common Terminology Criteria for Adverse Events, NCI CTCAE, Version 4.0). See table below:

Radiation Dermatitis Severity Score (RDS)

| Grade | Criteria/Characteristics |
|---|---|
| 0.0 | None. |
| 0.5 | Patchy faint follicular erythema; faint hyperpigmentation. |
| 1.0 | Faint diffuse erythema; diffuse hyperpigmentation. |
| 1.5 | Definite erythema; extreme darkening/hyperpigmentation. |
| 2.0 | Definite erythema (or hyperpigmentation) with fine desquamation and pruritis (itchiness). |
| 2.5 | Definite erythema (or hyperpigmentation) with branny desquamation. |
| 3.0 | Deep red erythema, diffuse desquamation; some desquamation in sheets. |
| 3.5 | Violaceous erythema, diffuse desquamation in sheets, and patchy crusting. |
| 4.0 | Violaceous erythema, diffuse desquamation in sheets, patchy crusting, and superficial ulceration. |

A severity score of at least 2 occurred in the third or fourth week of daily photon beam radiation treatment. Each radiation treatment delivered between 1.8 and 2.2 gray of radiation. Subjects were assigned one of three treatments as itemized in the following table.

TOPICAL STUDY TREATMENT COMPOSITIONS

| STUDY TREATMENTS | TREAMENT INGREDIENTS |
|---|---|
| APIGENIN | Water (Purified), Glycerin, Apigenin, Hyaluronic acid, Vitamins |
| LOTION | B₃ and B₅, Hydrogenated Polyisobutene, Cetearyl Alcohol, |
| | Ceteareth 20, Macadamia Ternifolia (Nut) Oil, Dimethicone, |
| | Tocopheryl Acetate (Vitamin E), Stearoxytrimethylsilane, Stearyl |
| | Alcohol, Panthenol, Farnesol, Benzyl Alcohol, Phenoxyethanol, |
| | Acrylates/C10 30 Alkyl Acrylate Crosspolymer, Sodium |
| | Hydroxide, Citric Acid |
| CONTROL | Water (Purified), Glycerin, Hydrogenated Polyisobutene, |
| LOTION | Cetearyl Alcohol, Ceteareth 20, Macadamia Ternifolia (Nut) |
| | Oil, Dimethicone, Tocopheryl Acetate (Vitamin E), |
| | Stearoxytrimethylsilane, Stearyl Alcohol, Panthenol, Farnesol, |
| | Benzyl Alcohol, Phenoxyethanol, Acrylates/C10 30 Alkyl |
| | Acrylate Crosspolymer, Sodium Hydroxide, Citric Acid |
| STANDARD OF | Wash the affected area with a mild soap and water rinse. Dry |
| CARE | thoroughly. |

Each treatment (Apigenin Lotion, Control Lotion, or Standard of Care) was performed 3 times per day. For those subjects assigned a lotion, they were told to apply a thin coat over the affected area. No bandages or coverings were applied for any of the treatments. Daily radiation treatment continued throughout the study unless otherwise noted.

The following Tables summarize the number and types of “Treatment Areas” as a function of “Days of Radiation Treatment” for the 3 “Study Treatments.”

TABLE 1
RADIATION TREATMENT WITH THE “BASE”
TOPICAL LOTION

| NUMBER OF | | DAYS OF RADIATION TREAMENT | | | | |
|---|---|---|---|---|---|---|
| TREATMENT AREAS | | 5 | 7 | 9 | 10 | 12 |
| 5 | BREASTS | 1 | 2 | 1 | — | 1 |
| 1 | BREAST UNDER | 1 | — | — | — | — |
| 2 | CERVIXES | — | — | — | 2 | — |

TABLE 2
RADIATION TREATMENT WITH THE “ESTANDARD
OF CARE” TOPICAL LOTION

| NUMBER OF | | DAYS OF RADIATION TREAMENT | | | | |
|---|---|---|---|---|---|---|
| TREATMENT AREAS | | 6 | 7 | 8 | 9 | 10 |
| 7 | BREASTS | — | — | 1 | 3 | 3 |
| 1 | BREAST UNDER | — | 1 | — | — | — |
| 2 | CERVIXES | 1 | — | — | — | 1 |

TABLE 3
RADIATION TREATMENT WITH THE “APIGENIN”
TOPICAL LOTION

| NUMBER OF | | DAYS OF RADIATION TREATMENT | | |
|---|---|---|---|---|
| TREATMENT AREAS | | 7 | 8 | 10 |
| 7 | BREASTS | — | 5 | 2 |
| 1 | BREAST UNDER | — | — | 10 |
| 2 | CERVIXES | 2 | — | |

### Experimental Results

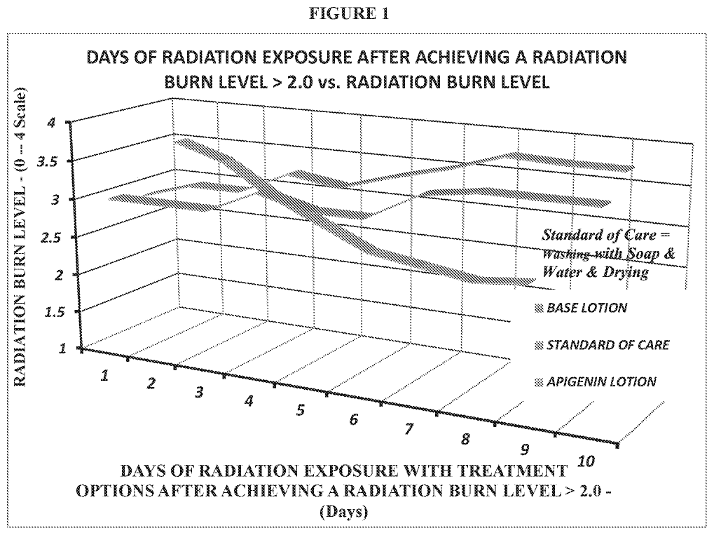

FIG. 1 is graph of the experimental data comparing the 3 treatments for the management of radiation dermatitis. The data indicates the positive outcomes for the apigenin containing moisturizing lotion in comparison to the “Control” moisturizing lotion and the “Standard of Care” treatment which consists of washing of the irradiated skin surface with a mild soap and water with subsequent drying. Of particular note is the improvement noted following application of the apigenin lotion after about the 4th day of radiotherapy treatment. In all cases, subjects treated with the apigenin lotion experienced “cleaner and dryer lesions” and a decided improvement in pain levels.

The experimental data indicate the following: The apigenin lotion is the only lotion that results in an improvement in the “Burning Scale” after about the 4th day of radiation treatment for “Breast”, “Breast (under)” and “Cervix” cancers. A significant reduction from a “Burn Level” of ˜3.5 to ˜2 represents an improvement of ˜40% after 6 additional days of radiation exposure.
In view of the wide range of radiation variables present in the 28 individuals exposed to radiotherapy therapy, the data clearly indicates the therapeutic superiority of the apigenin lotion over the “Base Lotion” and “Standard of Care” treatments.
Of particular note are the comments of the patients when referring to the apigenin lotion; i.e., the lesions in all cases are dryer, cleaner and as a result, pain level has improved.
The improved dryness is attributable to the occlusive film of the high molecular weight hyaluronic acid which functions as a sponge to retain water within its structural polysaccharide matrix and additionally serves to limit water loss via “trans-epidermal water loss.” In other words, moisture levels are retained within the HA occlusive film while the sponge-like properties of this occlusive film maintains a “dry” but water containing reservoir.

### Example 11

### Treatment of Thermal Burn

A young woman received 2^{nd} degree burns on her arm. After application of the nano-particulate apigenin lotion of Example 9 daily for nearly 2 weeks, the skin damage to the arm was remarkably reduced. There was no scarring as a result of her 2^{nd} degree burn.

### Example 12

### Effect on Chemically Induced Dermatitis in Standard Animal Model

### Background and Objective:

Dinitrochlorobenzene (DNCB) induction of dermatitis in mice or rats is a validated model for human atopic dermatitis and allergic dermatitis. While the mechanism of induction of dermatitis by DNCB differs from that of radiation dermatitis, the end result has many similarities and a key common feature is the disruption of the integrity of the skin with loss of stratum corneum and—to varying degrees—epidermis, exposing the dermis and basal layers of the skin and with deficits in the regular cycle of skin regeneration that occurs for normal, non-pathologic skin.

The present study was conducted to investigate which, if any, of several solubilized apigenin complexes demonstrated efficacy in promoting the resolution of dermatitis once formed.

### Methods of Procedure:

Animals: 95 BALB/c mice were supplied by the test facility, MB Research in Hatfield, Pa.

DNCB: This was also supplied by the test facility. For application it was at either 1% or 0.2% in ethanol.

### Overview of Test:

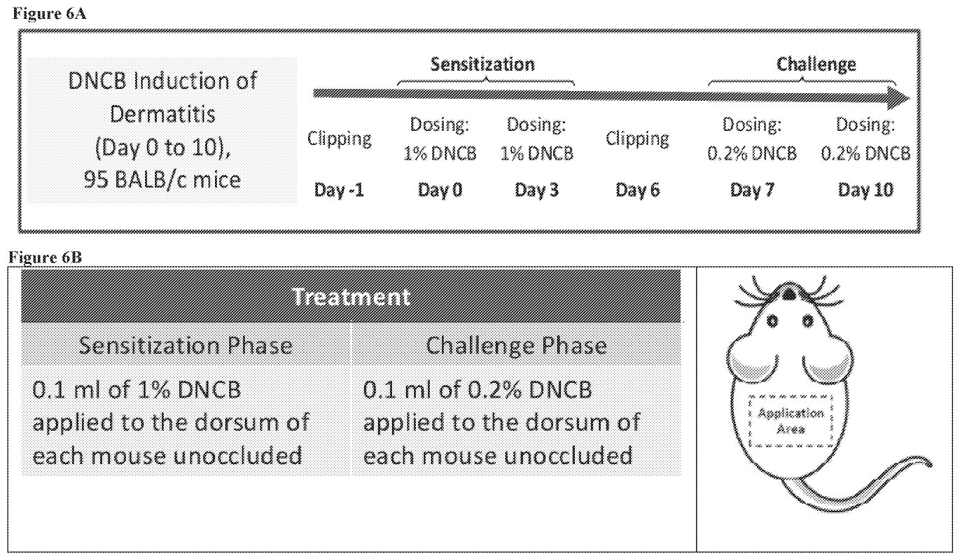

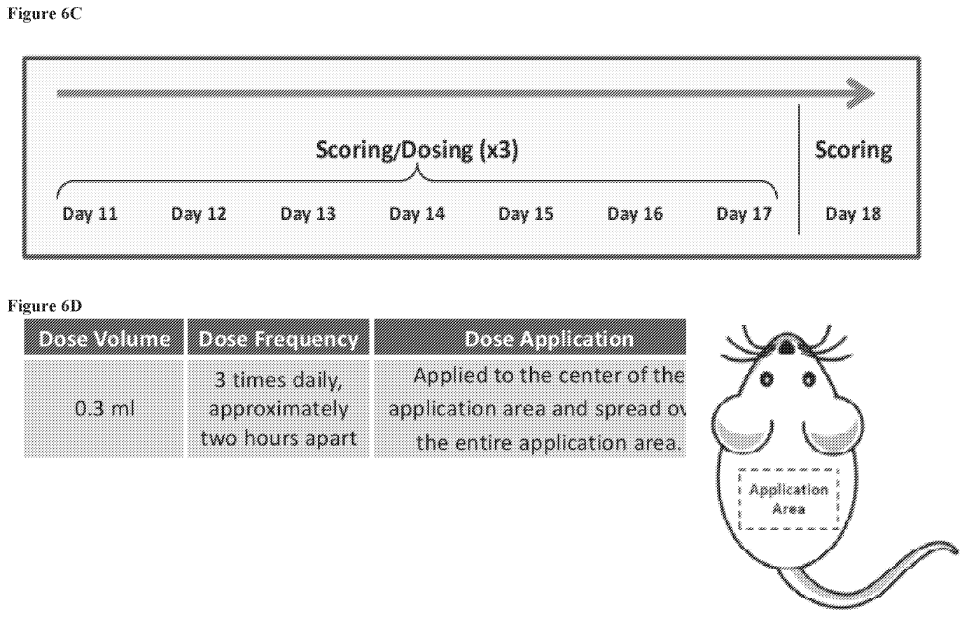

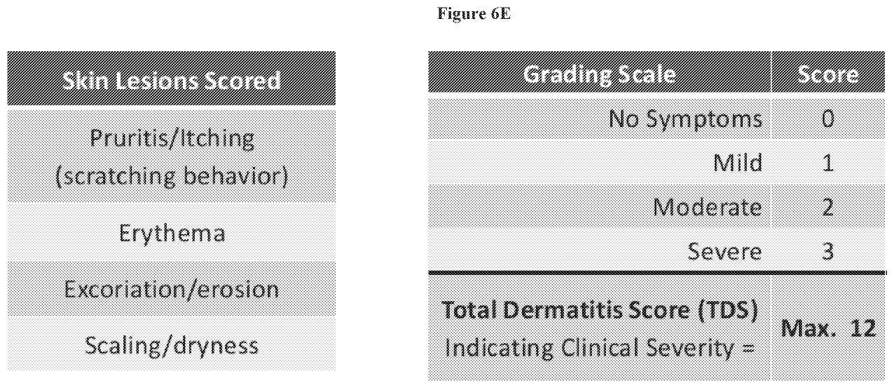

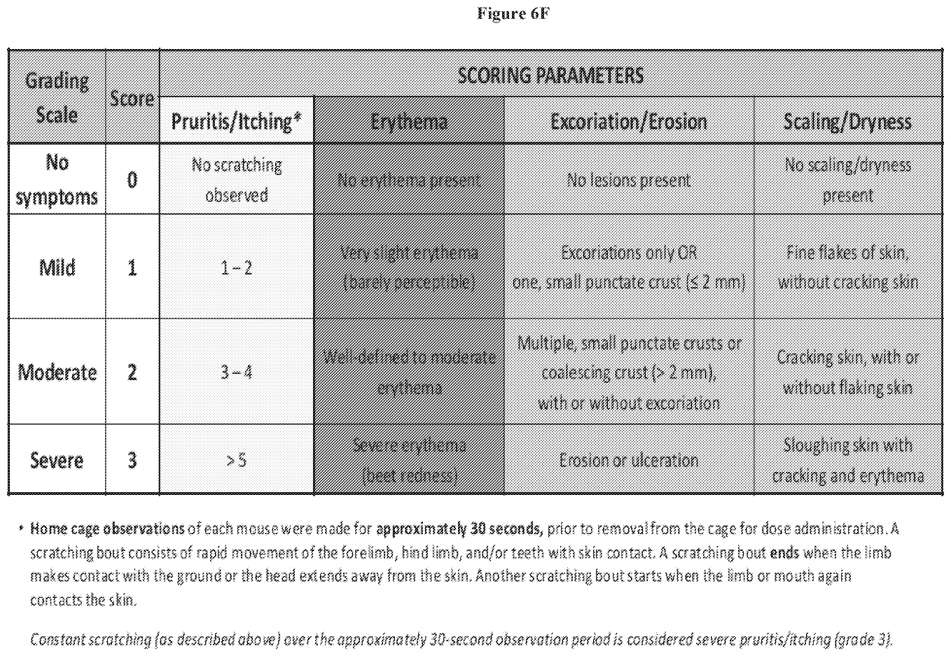

The procedure for conduct of the study is provided in FIG. 6A. During the induction of dermatitis phase the application procedures were as shown in FIG. 6B.

In the treatment and scoring phase the procedures were as shown in FIG. 6C. On each of Days 11 through 17 test articles were applied per the schedule shown in FIG. 6D.

This schedule provided for 7 days of treatment with test articles. Final scoring was on Day 18. The dermatitis and skin conditions grading systems are shown in FIG. 6E with the scoring parameters shown in FIG. 6F.

### Histopathology (Test #2 Only):

Skin samples from the Day 18 scoring were fixed for histopathology exam for microscopic scoring of skin inflammation and condition.

### Test Groups:

Unless otherwise noted, all apigenin containing formulations were 1.5% apigenin. The test groups in this study were as described in the “Results” section below. Two tests were conducted and are described below. “Viz base” is described in Ex. 8. “HA blend” (high and low MW HA) is described in Ex. 4. “In Situ lotion” is described in Ex. 7. “Vitamins” refers to niacin (B3) and panthenon (B5)

### Results

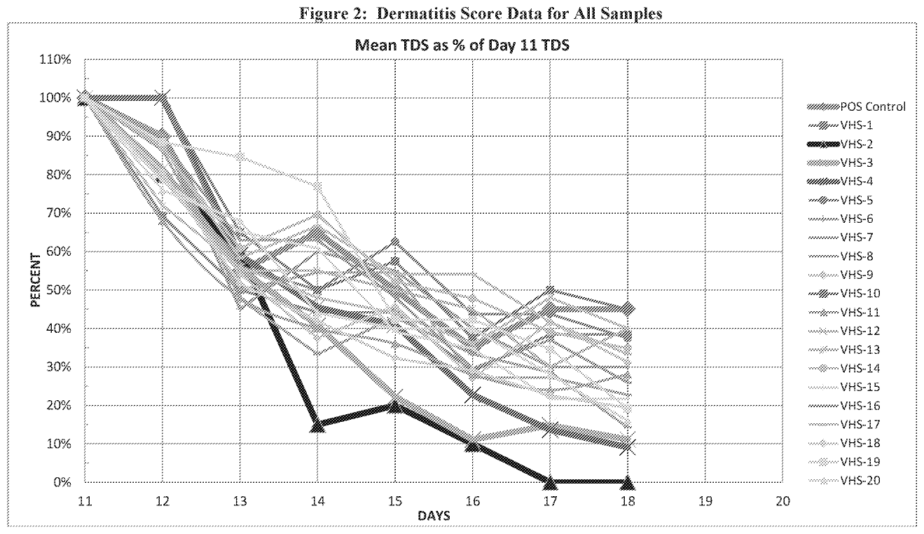

Dermatitis, Clinical Scoring: Dermatitis scores over the scoring period for each test article are provided in FIGS. 2-5.

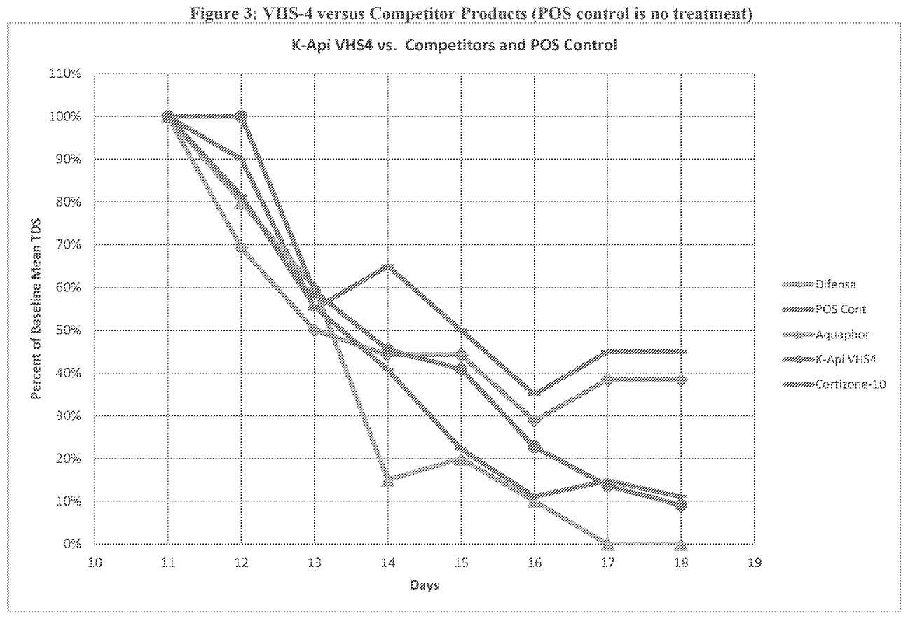

Key to Sample Identification for FIGS. 2 and 3

| Formulation Description | Sample ID |
|---|---|
| Untreated mouse/Control | POS Control |
| Difinsa 53/competitive product (pH = 6.10) | VHS-1 |
| Aquaphor/‘Standard of care’ product (pH = 5.49)/active- petrolatum 41% | VHS-2 |
| Cortizone-10/‘Standard of care’ product (pH = 5.20) | VHS-3 |
| K-Apigenin. ‘In-Situ’ lotion, Viz base, with HA blend, with vitamins | VHS-4 |
| Na-Apigenin., ‘In-Situ’ lotion, Viz base, with HA blend, with vitamins | VHS-5 |
| Hydrated nano Apigenin 90/10, ‘slow’ precipitation, Viz base, HA blend, | VHS-6 |
| vitamins, no sonication | |
| Na-Apigenin, In-Situ lotion, Viz base, no HA, with vitamins, no | VHS-7 |
| sonication | |
| Na-Apigenin, In-Situ lotion, Viz base, no HA, no vitamins, no sonication | VHS-8 |
| Unmodified raw apigenin., Viz base, with HA blend, original ‘PMC’ | VHS-9 |
| procedure w/sonication | |
| Unmodified raw apigenin., Viz base, HA blend, apigenin mixed w/cold | VHS-10 |
| PS80 then added to lotion, no sonication | |
| Hydrated nano Apigenin 90/10, Cetaphil base, HA blend, vitamins, | VHS-11 |
| original ‘PMC’ procedure w/sonication | |
| Hydrated nano Apigenin 90/10, Cetaphil base, HA blend, vitamins, no | VHS-12 |
| sonication | |
| Hydrated nano Apigenin 90/10, Viz base, HA blend, vitamins, no | VHS-13 |
| sonication | |
| Hydrated nano Apigenin 90/10, Viz base, no HA, vitamins, no sonication | VHS-14 |
| Hydrated nano Apigenin 90/10, Viz base, Low mol weight HA, vitamins, | VHS-15 |
| no sonication | |
| Hydrated nano Apigenin 90/10, Viz base, High mol weight HA, vitamins, | VHS-16 |
| no sonication | |
| Thermally solubilized Apigenin in PS80, Viz base, HA blend, vitamins, | VHS-17 |
| no sonication | |
| Lotion ‘blank’ - no apigenin, Viz base, HA blend, vit B3, panthenol | VHS-18 |
| Lotion ‘blank’ - no apigenin, Viz base, HA blend, vit B3, panthenol and | VHS-19 |
| vitamin C | |
| Na-Apigenin, In-Situ lotion, Viz base, HA blend, vitamins. Different | VHS-20 |
| order of addition: HA blend mixed into a lotion base, and then Na- | |
| Apigenin solution & citric acid were added | |

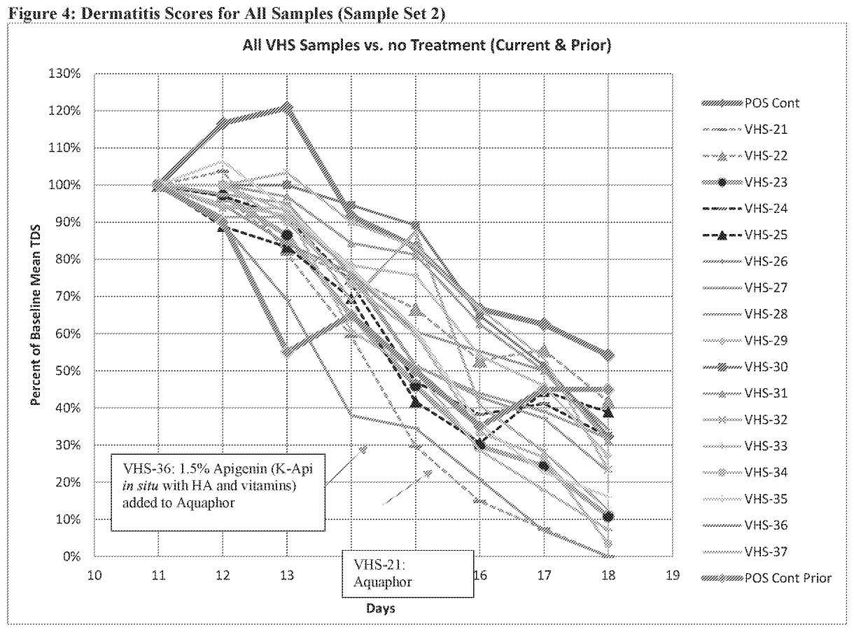

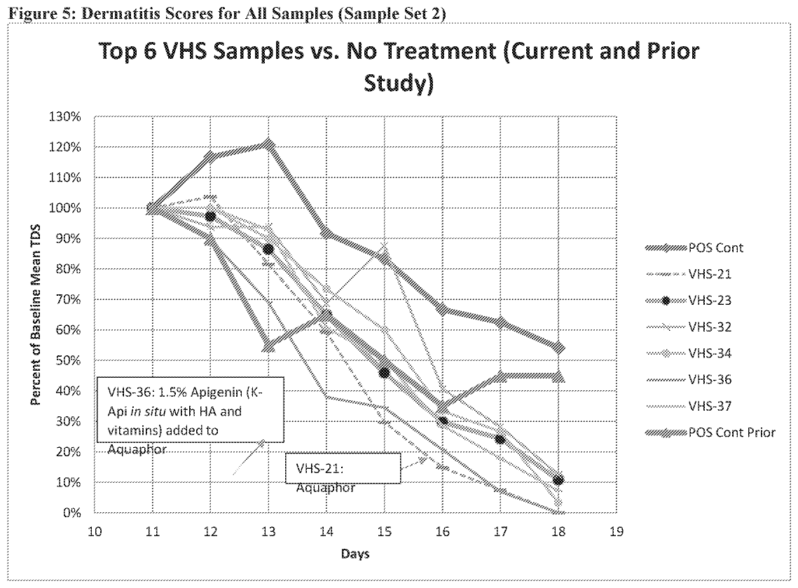

Key to Sample Identification for FIGS. 4 and 5

| Formulation Description | Sample ID |
|---|---|
| Untreated mouse/Control | POS Control |
| Aquaphor, commercial product, nothing added. This is “Standard of care” product | VHS-21 |
| Cortizone-10/‘Standard of care’ product (pH = 5.20), active- hydrocortisone 1% | VHS-22 |
| Aquaphor, with added 1.5% Apigenin nano paste 90/10, with HA, and vitamins | VHS-23 |
| Cetaphil Moisturizing Cream - commercial sample (no apigenin, no HA, no | VHS-24 |
| vitamins), pH = 4.76 | |
| Cetaphil Moisturizing. Cream -commercial sample with added 1.5% K-Apigenin | VHS-25 |
| In-Situ, no HA, no vit. | |
| K-Apigenin, 1.5% ‘In-Situ’ lotion, Viz base, HA blend, vitamins | VHS-26 |
| K-Apigenin, 1.5% ‘In-Situ’ lotion, Viz base, no HA, no vitamins | VHS-27 |
| Na-Apigenin, 1.5%, In-Situ lotion, Viz base, no HA, no vitamins | VHS-28 |
| Lotion ‘blank’ - no apigenin, Viz base, HA blend, vit B3, panthenol- 1% | VHS-29 |
| dimethicone | |
| Lotion ‘blank’ - no apigenin, Viz base, HA blend, vit B3, panthenol - 5% | VHS-30 |
| dimethicone | |
| Hydrated 0.75% nano-‘slow’ precipitated, Viz base, HA blend, vitamins | VHS-31 |
| Lotion with 0.75% Apigenin, as K-Apigenin In Situ, without HA and without | VHS-32 |
| vitamins | |
| Hydrated 0.75% nano-‘slow’ precipitated, Viz base, HA blend, vitamins | VHS-33 |
| Viz base lotion with 1.5% Combo apigenin (0.75 Nano and 0.75 Na-In Situ, with | VHS-34 |
| HA, vitamins | |
| Cream Base (higher lipid content) prototype, 1.5% K-Apigenin In-situ, HA blend, | VHS-35 |
| vitamins | |
| Aquaphor, with added 1.5% Apigenin, as K-Apigenin in Situ, with HA, and | VHS-36 |
| vitamins | |
| Viz base Lotion with 10% Apigenin, added as nano paste, nano paste 70/30 was | VHS-37 |
| used | |

### Histology of Day 18 Skin Samples:

Summary (from Histology Report)

Female mice with DNCB-induced dermatitis were administered one of 17 test articles (VHS-21, VHS-22, VHS-23, VHS-24, VHS-25, VHS-26, VHS-27, VHS-28, VHS-29, VHS-30, VHS-31, VHS-32, VHS-33, VHS-34, VHS-35, VHS-36 or VHS-37) three times a day at a dose level of 0.3 ml/site for seven days. This resulted in varying degrees of resolution of the inflammation. Three test articles showed a slight increase in the inflammation score when compared to the positive control (VHS-24, VHS-25 and VHS-29) and two showed little difference from the positive control (VHS-22 and VHS-26). The four test articles that showed the most notable improvement in inflammation score (resulting in >30% decrease in score compared to the positive control) were VHS-36, VHS-37, VHS-35, and VHS-21. The greatest resolution (59.1%) was seen with VHS-36.

Each skin section was evaluated for microscopic changes. Changes present were graded (1=minimal, 2=mild, 3=moderate, 4=marked, 5=severe). All numeric diagnoses were summed for each group to give an inflammation score as shown below. The difference between the group inflammation score and the score for the positive control group was calculated and was used to calculate a ranking of the test articles. Ranks in parentheses indicate an inflammation score greater than that seen in the positive control group.

Summary of Inflammation Scores and Resolution

| | | | Percent | |
|---|---|---|---|---|
| | | | Difference | |
| | | Inflammation | From Positive | |
| Group | Test Article | Score | Control | Rank |
| 1 | Negative Control | 0 | −100 | N/A |
| | (Sham) | | | |
| 2 | Positive Control | 115 | 0 | N/A |
| 3 | VHS-21¹ | 65 | 43.5 | 4 |
| 4 | VHS-22 | 112 | −2.6 | 13 |
| 5 | VHS-23 | 82 | −28.7 | 5 |
| 6 | VHS-24 | 132 | 14.8 | (17) |
| 7 | VHS-25 | 127 | 10.4 | (16) |
| 8 | VHS-26 | 117 | 1.7 | (14) |
| 9 | VHS-27 | 105 | −8.7 | 12 |
| 10 | VHS-28 | 104 | −9.6 | 11 |
| 11 | VHS-29 | 119 | 3.5 | (15) |
| 12 | VHS-30 | 102 | −11.3 | 10 |
| 13 | VHS-31 | 85 | −26.1 | 6 |
| 14 | VHS-32 | 94 | −18.3 | 7 |
| 15 | VHS-33 | 97 | −15.7 | 8 |
| 16 | VHS-34 | 100 | −13.0 | 9 |
| 17 | VHS-35 | 74 | −35.7 | 3 |
| 18 | VHS-36² | 47 | −59.1 | 1 |
| 19 | VHS-37 | 60 | −47.8 | 2 |

¹Aquaphor
²1.5% K-Apigenin in situ nano with HA and vitamins added to Aquaphor

Microscopic changes seen included, but were not limited to, hyperplastic changes in the epidermis and adnexa, inflammation in the dermis, dermal fibroplasia and adnexal arrest or loss.

### DISCUSSION

Two studies were conducted using the DNCB-induced dermatitis model in 95 BALBc mice. These studies were designed to investigate the effect of the test materials on healing of dermatitis once formed. Positive control in each study was no treatment of the affected area. Competitor controls in the first study were Aquaphor (a common standard of care product that is 50% petrolatum), Cortizone-10 (a corticosteroid preparation containing 1% hydrocortisone), and Difensa53 (being promoted by its manufacturer as effective for management of radiation dermatitis and which contains silybin (from milk thistle). In the second study, Cortisone 10 and Difinsa53 were dropped as competitor controls.

In both studies, clinically scored dermatitis severity was assessed daily after induction of dermatitis by DNCB, which peaked at Day 11 in each study. During the assessment phase test products were applied 3-times a day to the affected area (on Days 11-17). A final assessment was conducted on Day 18.

In the second study, skin samples were fixed and examined microscopically for scoring of microscopic skin inflammation. The purpose of this as to investigate whether the end-stage skin condition varied from test product to test product.

In the clinical assessments in both studies, Aquaphor performed consistently very well and generally resulted on complete clinical clearing by Day 18. Difinsa53 did not perform well in the clinical assessments of the first study and did no better than no treatment (POS Control). Cortisone 10 performed well in the first study but did not provide as much clearing by Day 18 as did Aquaphor (89% clearing vs. 100% clearing, respectively). However, the difference in performance was not statistically significant.

In the first study, the best performance from an apigenin containing test article was provided by VHS-4. This was K-Apigenin ‘In-Situ’ lotion, in the Viz base, with LMW/HMW HA blend and vitamins. This provided 91% clearing by Day 18, which was not statistically significantly different from Aquaphor.

In the second study, Aquaphor and 1.5% K-Apigenin with HA and vitamins in Aquaphor both performed equally well in clinical assessments. However, the microscopic skin condition was better for the K-Apigenin-Aquaphor blend than for Aquaphor alone.

In a fractionated dose regimen, there are intervals where the skin is trying to recover from the preceding exposure. The effect of the apigenin preparation on promoting healing improves the degree of skin healing that occurs between fractionated dose treatments.

### Example 13

### Comparison of Nano-Apigenin Paste Produced by Standard Filtration and without Filtration and In Situ Method

### Objective:

To compare the fiber sizes in the morphology of Nano-apigenin paste prepared by Standard Filtration and Without Filtration processes

### SUMMARY

This study was conducted to compare the morphology of Nano-apigenin paste prepared using the long Standard Filtration process with very short Without Filtration process. Four Nano-apigenin paste prepared by Standard Filtration and Without Filtration were sent to the University of Maryland Imaging Center for transition electron microscopy (TEM) examination. In addition, 3 different lotions containing Nano-apigenin paste from Standard Filtration and Without Filtration as well as a placebo lotion were sent to the center. Estimation of the fiber sizes from the TEM micrographs of the samples produced by Standard Filtration and Without Filtration did not reveal any significant differences between the Nano-apigenin fibers. The sizes of the fibers produced either by Standard Filtration or Without Filtration were less than 400 μm. The texture of the Nano-apigenin paste from Without Filtration process is very smooth.

### INTRODUCTION

Nano-apigenin paste in water can be prepared and filtered under vacuum to afford 10% apigenin and 90% water. During filtration the moisture content is checked periodically until 90% moisture content was achieved. The process is referred to as “Standard Filtration”. The paste is then added to the lotion formulation to give 1.5% Nano-apigenin lotion. In an alternative process of making the Nano-apigenin paste, 10% Nano-apigenin paste was prepared without filtration. This process is referred to as “Without Filtration”.

### Methods and Materials:

All chemicals used in this analysis were HPLC grade and were purchased from Sigma-Aldrich (St Louis, Mo.).

Standard Filtration: Weigh 48.0 g (0.18 mole) of apigenin into a 2-Liter beaker and add 1000 mL of DI water. Stir the mixture and add 28.2 g of 50% sodium hydroxide solution. Continue stirring until all apigenin is completely dissolved. Transfer 135.3 g of 50% citric acid solution into an addition funnel and slowly add it to the sodium apigenin solution. Continue to stir for 60 min. Analyze the wet apigenin paste for moisture content with Sartorius Moisture Analyzer (The Scale People Inc., Columbia, Md.).

Subject the ensuing mixture to vacuum filtration using coarse filter sheet by GE Healthcare Whatman. After 10 min recheck the moisture content of the apigenin cake. Continue moisture analysis and filtration until the desired percentage is achieved.

Without Filtration: Weigh 45.8 g DI water into a 250-mL beaker. Weigh 3.2 g of NaOH pellets and add to the beaker and stir until all dissolve. (Note: the temperature of water will increase due to heat of dissolution). Weigh 10.2 g apigenin powder and slowly add to the beaker over 5 min. Continue stirring until all apigenin is completely dissolved about 20 min. Transfer the sodium apigenin solution into an addition funnel. Rinse the beaker with 5 mL of water and add to the addition funnel. Weigh 30.8 g of 50% citric acid solution into a 250-mL beaker. Place underneath the mechanical stirrer and stir. Add sodium apigenin solution slowly over 10 min with continuous stirring. Rinse the addition funnel with the remaining 5 mL of water. Continue stirring for additional 60 min. Record the final pH (pH=4.5-4.7).

IN-SITU Generation of 1.5% Nano-Apigenin Paste in Lotion (5000 g): Weigh 30 g of 25% sodium apigenin solution and add slowly to the lotion during 5 min and stir the mixture for 15 min. Record the pH (pH≤9.5). Leave the pH probe in the lotion avoiding contact with the mechanical stirrer. Weigh 23.8 g of 50% citric acid solution and transfer to an addition funnel. Add slowly to the mixture with vigorous stirring while observing the pH changes and stop when the pH is between 4.5-4.7.

Scanning Electron Microscopy: The transition electron microscopy was performed on FEI Quanta 200 under low vacuum with tungsten electron source. The samples were applied onto cryo TEM stubs and flash frozen in liquid nitrogen. Frozen specimen were then transferred in the vacuum chamber of the cryo-transfer system Alto 2100 (Gatan, UK) at ˜−160° C. and fractured using a sharp blade cooled at liquid nitrogen temperature. Specimen with fractured surface were transferred into Quanta 200 TEM (Thermo Fisher, formerly FEI Co.) fitted with a cryo stage at −120° C. and sublimated by warming up the stage to −100° C. and maintained for 5 min to remove surface water. The etched surface were then sputtered coated with 10-20 nm gold to enhance conductivity of the specimen and Imaged at 5 keV at −150° C. in the cryo TEM.

Transition Electron Microscopy: About 0.25 gram of each emulsified specimen was transferred into a 1.5 ml microcentrifuge tube, washed with 50% ethanol three times and with pure water two times. Specimen were centrifuged at 13 K rpm, 15 min between each wash. Pellet from the final wash was re-suspended into 100 microliter deionized water. 5 to 10 microliter of the suspension were applied onto 400 mesh formvar coated copper grids, rinsed with water, negatively stained with 1% uranyl acetate, air dried and examined in a transmission electron microscope (Tecnai T12, FEI) operated at 80 kV. Digital images were acquired using an AMT bottom mount CCD camera and AMT600 software.

The samples submitted for TEM analysis are given in the table below.

| TEM | | Method of Nano-Apigenin |
|---|---|---|
| ID. | Sample | preparation |
| A | 10% Nano-Apigenin paste | Standard Filtration |
| B | 10% Nano-Apigenin paste | Without Filtration |
| C | 15% Nano-Apigenin paste | Without Filtration |
| D | Lotion containing 3.0% | 1.5% Nano-Apigenin paste |
| | Nano-Apigenin paste | from Standard Filtration |
| | | 1.5% from IN-SITU* |
| E | PMC containing 1.5% | Standard Filtration |
| | Nano-Apigenin | |
| F | Lotion containing 1.5% | Without Filtration |
| | Nano-Apigenin⁵ | |
| G | Lotion containing 3.0% | 1.5% Nano-Apigenin paste |
| | Nano-Apigenin paste⁶ | from Without Filtration |
| | | 1.5% from IN-SITU |
| H | Placebo Lotion | Without added Nano-Apigenin |

*Nano-apigenin is generated in the lotion

### Results and Discussion:

Initially, the samples were analyzed on Scanning Electron Microscope (SEM), however, the micrographs did not clearly reveal the fibers there by making it difficult to accurately measure the sizes. In view of this it was envisaged to perform Transition Electron Microscope (TEM). The 10% Nano-Apigenin paste (Sample B) from Without Filtration process was not analyzed in lieu of the 15% paste (Sample C). In the case of the lotion without added apigenin (Sample H; placebo), TEM was not recorded since no fibers were detected in SEM. The size of the fibers were estimated base on lens magnification. The yellow line in the micrograph indicates the width of the fiber measured. Not less than 50 fibers were measured for each sample. The fiber sizes range from 18 um to 370 μm. The sizes of the fibers are summarized in the table below.

Fiber size (μm) of Nano-apigenin paste

| | | Approximate |
|---|---|---|
| TEM | | Fiber size |
| ID. | Sample | (μm) |
| A | 10% Nano-Apigenin paste from Standard Filtration | 18-369 |
| B | 10% Nano-Apigenin paste from Without Filtration | NA* |
| C | 15% Nano-Apigenin paste from Without Filtration | 15-116 |
| D | Lotion containing 3.0% Nano-Apigenin paste: 1.5% | 18-305 |
| | from Standard Filtration and 1.5% from IN-SITU | |
| E | PMC containing 1.5% Nano-Apigenin from | 38-211 |
| | Standard Filtration | |
| F | Lotion containing 1.5% Nano-Apigenin from | 18-217 |
| | Without Filtration | |
| G | Lotion containing 3.0% Nano-Apigenin paste: 1.5% | 12-173 |
| | from Without Filtration and 1.5% from IN-SITU | |
| H | Placebo Lotion (Without Nano-apigenin paste) | Not detected |

*NA = Not Available

### CONCLUSION

The TEM micrographs of the Nano-apigenin paste revealed that there was no significant difference between the sizes of the fibers produced by Standard Filtration procedure and those of Without Filtration procedure. The sizes of the fibers produced either by Standard Filtration or Without Filtration were less than 400 μm.

It should be understood that a wide range of changes and modifications could be made to the embodiments described above. It is therefore intended that the foregoing description illustrates rather than limits this invention, and that it is the following claims, including all equivalents, which define this invention.

What is claimed:

1. A composition for topical administration comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
wherein the composition is greater than 70% water, or water and petrolatum, and has a pH of 3-8.

2. The composition of claim 1, wherein said polyphenol is one or more compounds selected from the group consisting of a phenolic acid, a flavonoid, a stibene, a diferuloylmethane, a tannin, and a lignan.

3. The composition of claim 1, wherein said polyphenol is a flavonoid and is one or more compounds selected from the group consisting of a flavonol, a flavanone, a flavanol, a flavone, an anthocyanin, and an isoflavone.

4. The composition of claim 1, wherein said polyphenol is one or more compounds selected from the group consisting of apigenin, quercetin, luteolin, rutin, naringenin, morin, genistein, kaempferol, curcumin, resveratrol, EGCG and a catechin.

5. The composition of claim 1, wherein the hyaluronic acid is a mixture of:
40-90% by wt. of hyaluronic acid having a molecular weight of 8000-15000 D, and
10-60% by wt. of hyaluronic acid having a molecular weight of 800,000-1.2 million D.

6. The composition of claim 1, wherein the composition comprises 78-85% water, or water and petrolatum.

7. The composition of claim 1, wherein the composition comprises 81-88% water, or water and petrolatum.

8. The composition of claim 1, wherein the composition has a pH of 4-7.

9. The composition of claim 1, wherein said dermatologically acceptable carrier includes petrolatum.

10. The composition of claim 1, wherein said dermatologically acceptable carrier includes colloidal oatmeal.

11. A composition as in claim 1 wherein said dermatologically acceptable carrier includes petrolatum and colloidal oatmeal.

12. The composition of claim 1, wherein said dermatologically acceptable carrier includes one or more vitamins selected from the group consisting of vitamin B3 and vitamin B5.

13. The composition of claim 1, wherein said dermatologically acceptable carrier includes a DNA repair agent.

14. A method of treating radiation dermatitis in a mammal comprising administering topically to the affected area of skin of said mammal a therapeutically effective amount of a composition comprising:
iv) 0.01-15% by wt. polyphenol in a form selected from the group consisting of a concentrate, a salt, and a nanoparticle,
v) 0.01-5% by wt. hyaluronic acid, wherein the hyaluronic acid includes a high molecular weight hyaluronic acid and a low molecular weight hyaluronic acid, and
vi) greater than 70% by wt. of a dermatologically acceptable carrier,
wherein the composition is greater than 70% water, or water and petrolatum, and has a pH of 3-8.

15. The method of claim 14, wherein said composition is administered 2 or 3 times per day.

16. The method of claim 14, wherein said radiation dermatitis is acute radiation dermatitis.

17. The method of claim 14, wherein said radiation dermatitis is chronic radiation dermatitis.

18. The method of claim 14, wherein said radiation dermatitis is dry desquamation.

19. The method of claim 14, wherein said radiation dermatitis is moist desquamation.

20. A method of treating eczema in a mammal comprising administering topically to the affected area of skin of said mammal a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
wherein the composition is greater than 70% water, or water and petrolatum, and has a pH of 3-8.

21. The method of claim 20, wherein said composition is administered 2 or 3 times per day.

22. The method of claim 20, wherein said eczema is selected from the group consisting of atopic dermatitis, stasis dermatitis, scabies, fungal dermatitis, pompholyx, nummular eczema, lichen simplex chronicus, seborrheic dermatitis, xerotic eczema, and allergic contact dermatitis.

23. A method of treating a tumor in a mammal comprising:
c) administering a therapeutically effective dose of ionizing radiation to a tumor in said mammal, and
d) applying to the affected area of the skin a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
wherein the composition is greater than 70% water, or water and petrolatum, and has a pH of 3-8.

24. The method of claim 23, wherein said ionizing radiation is selected from the group consisting of alpha radiation, beta radiation, X-rays, gamma radiation, and flouroscopic radiation.

25. The method of claim 23, wherein said radiation is administered 1-7 times per week.

26. The method of claim 23, wherein said radiation is administered at dosages of 1-3 Gray per treatment.

27. The method of claim 23, wherein said composition is administered 2 or 3 times per day.

28. A method of treating a thermal burn in a mammal comprising administering topically to the affected area of skin of said mammal a therapeutically effective amount of a composition comprising
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid and a low molecular weight hyaluronic acid, and
iii) greater than 70% by wt. of a dermatologically acceptable carrier,
wherein the composition is greater than 70% water, or water and petrolatum, and has a pH of 3-8.

29. The method of claim 28, wherein said thermal burn is a first degree burn.

30. A method of treating a wound in a mammal comprising:
administering topically to the wound of said mammal a therapeutically effective amount of a composition comprising:
iv) 0.01-15% by wt. polyphenol in a form selected from the group consisting of a concentrate, a salt, and a nanoparticle,
v) 0.01-5% by wt. hyaluronic acid, wherein the hyaluronic acid includes a high molecular weight hyaluronic acid and a low molecular weight hyaluronic acid, and
vi) greater than 70% by wt. of a dermatologically acceptable carrier,
wherein the composition is greater than 70% water, or water and petrolatum, and has a pH of 3-8.

31. The method of claim 30, wherein the pH of the composition is less than 5.

32. A method of treating a nonmelanoma skin cancer or xeroderma pigmentosum in a mammal comprising administering topically to the affected area of skin of said mammal a therapeutically effective amount of a composition comprising:
i) 0.01-15% by wt. polyphenol in a form selected from the group consisting of a concentrate, a salt, and a nanoparticle,
ii) 0.01-5% by wt. hyaluronic acid, wherein the hyaluronic acid includes a high molecular weight hyaluronic acid as well as a low molecular weight hyaluronic acid,
iii) 0.5-5% by wt. 5-fluorouracil, and
iv) greater than 70% by wt. of a dermatologically acceptable carrier,
wherein the composition is greater than 70% water, or water and petrolatum, and has a pH of 3-8.
